(19) United States
(12) Patent Application Publication
(10) Pub. No.: US 2026/0335537 A1
(43) Pub. Date: Oct. 29, 2026

(54) MEDICAL PUMP

(71) Applicant: DEKA Products Limited Partnership, Manchester, NH (US)

(72) Inventors: Jonathan Parker, Henniker, NH (US); Jonathan R. Thurber, Deerfield, NH (US)

(21) Appl. No.: 19/725,231

(22) Filed: Jun. 30, 2026

Related U.S. Application Data
(63) Continuation of application No. 18/198,905, filed on May 18, 2023.
Application No. 18/198,905 is a continuation-in-part of application No. 16/542,789, filed on Aug. 16, 2019, now Pat. No. 11,707,615.
(60) Provisional application No. 62/765,100, filed on Aug. 16, 2018.
(60) Provisional application No. 63/832,834, filed on Jun. 30, 2025.

(51) Int. Cl. A61M 5/142; A61M 5/145

(52) U.S. Cl. CPC A61M 5/14228; A61M 5/1452; A61M 2005/14533

(57) ABSTRACT

An ultrasonic monitoring system may detect air in a fluid line while identifying acoustic bridging external to an internal lumen of the line. A transducer arrangement may be operated under a sensing condition to obtain response data for air-in-line detection and under a bridge-diagnostic condition to obtain diagnostic response data. A controller may determine that an external acoustic bridge exists when the diagnostic response data satisfy a bridge criterion. Because the bridge may carry ultrasonic energy along or around an exterior surface of the conduit rather than through the fluid path, the controller may generate an alarm condition indicating that air-in-line detection is unreliable and may prevent a fluid-present response from being used as a valid air-clear indication while the alarm remains active. The system may be integrated with a medical pump and may use calibration data, diagnostic windows, bridge-clearing tests, set identification, pump inhibition, bubble-volume estimation, pressure sensing, delivered-volume compensation, and diagnostic logging functions.

### CROSS-REFERENCE TO RELATED APPLICATIONS

This application is a continuation-in-part of U.S. patent application Ser. No. 18/198,905, filed May 18, 2023, entitled “Medical Pump,” published as U.S. Patent Application Publication No. 2023/0285662A1, which is a continuation-in-part of U.S. patent application Ser. No. 16/542,789, filed Aug. 16, 2019, now U.S. Pat. No. 11,707,615, and which claims the benefit of U.S. Provisional Patent Application No. 62/765,100, filed Aug. 16, 2018. This application also claims the benefit of U.S. Provisional Patent Application No. 63/832,834, filed Jun. 30, 2025, entitled “Medical Pump.” The entire disclosure of each of the foregoing applications is incorporated herein by reference.

### BACKGROUND

### Technical Field

The present disclosure relates generally to medical fluid delivery systems and, more particularly, to ultrasonic air-in-line detection, fluid-presence sensing, diagnostic signal processing, calibration, contamination detection, and pump-control systems for medical fluid conduits.

### Description of Related Art

In many medical settings, fluid is delivered to a patient through a fluid line. Depending on the therapy, a clinical user may need to deliver fluid rapidly, accurately, intermittently, or slowly over an extended time. Medical fluid delivery may include intravenous infusion, transfusion, dialysis-related therapies, enteral or parenteral nutrition, bolus delivery, fluid resuscitation, insulin delivery, and other therapies involving a flexible or rigid fluid conduit.

Air may enter or form in a fluid line. Air bubbles may be introduced during priming, set loading, reservoir connection or disconnection, outgassing, agitation, changes in pressure, changes in temperature, or other operating conditions. Excessive air delivered to a patient may be detrimental. Air in a fluid line may also displace therapeutic fluid and therefore may affect delivered-volume estimates.

Fluid delivery may be performed using a gravity-fed line, an infusion pump, or another medical fluid device. A gravity-fed system may include a fluid reservoir, such as an IV bag, connected to a fluid line and a drip chamber. In some circumstances, flow may be manually adjusted. In other circumstances, strict flow control is required and a programmable infusion pump may be used. Air-in-line detection may be useful in both gravity-fed and pump-assisted fluid delivery systems.

Ultrasonic sensing may be used to detect air or fluid in a conduit by transmitting acoustic energy through or near the conduit and measuring energy received by a receiver. In general, liquid in the conduit may acoustically couple a transmitter and a receiver more effectively than air in the conduit. However, external acoustic paths may make an ultrasonic air detector unreliable if such external acoustic paths are not detected, rejected, or otherwise accounted for.

For example, moisture, water films, gel, residue, cleaning solution, condensation, spilled fluid, or other contaminants may create an acoustic bridge along or around an exterior surface of a fluid conduit. Such bridging may cause ultrasonic energy to bypass the intended through-lumen path and travel around the outside of the tube instead of through the fluid or air in the internal lumen. If not recognized, the bypass path may mask an air-in-line condition, may make a tube appear fluid-filled when air or a bubble is present, or may otherwise make the ultrasonic air detector unreliable. Accordingly, detected bridging may itself represent an alarmable sensor-fault or air-detection-unreliable condition. Sensor-to-sensor acoustic variation, tubing material, tubing position, signal offset, electronic drift, acoustic ringing, line pressure, and mechanical tolerance stack-up may also affect signal reliability.

The present disclosure addresses these and other technical issues by providing ultrasonic sensing architectures, quadrature demodulation and active-rectification circuits, per-unit calibration, frequency-sweep diagnostics, off-band bridge detection, multi-threshold air-event characterization, multiple acoustic path sensing, air-volume estimation, bridge-alarm generation, pump inhibition, and mechanical structures for repeatable conduit positioning. This background discussion is provided to explain technical context and is not an admission that any statement herein is prior art.

### SUMMARY

In some embodiments, a fluid-line monitoring system may include a fluid conduit having an internal lumen, an ultrasonic transducer arrangement positioned relative to the fluid conduit, a signal-generation circuit configured to operate the ultrasonic transducer arrangement under a first sensing condition and under a second diagnostic condition different from the first sensing condition, a signal-processing circuit configured to generate ultrasonic response data responsive to ultrasonic energy received by the ultrasonic transducer arrangement, and a controller configured to evaluate the ultrasonic response data.

In some embodiments, the controller may obtain first response data while the ultrasonic transducer arrangement is operated under a sensing condition for air-in-line detection, obtain diagnostic response data while the ultrasonic transducer arrangement is operated under a bridge-diagnostic condition, determine an external-bridge condition based on the diagnostic response data satisfying a bridge criterion, and generate an alarm condition indicating that air detection by the ultrasonic transducer arrangement is unreliable. The alarm condition may be generated because the ultrasonic signal may be traveling along or around an exterior surface of the tube rather than through the internal lumen, such that bubbles in the tube may not be reliably detected. In some embodiments, the controller may prevent a fluid-present response from being used as a valid air-clear indication while the alarm condition remains active.

In some embodiments, the first sensing condition may correspond to a normal air/fluid sensing condition, and the second diagnostic condition may correspond to a bridge-diagnostic condition. The second diagnostic condition may include a diagnostic frequency different from an operating frequency, a plurality of diagnostic frequencies, a frequency sweep, a chirp, a coded excitation, a phase-coherent excitation, a diagnostic sample window, a ring-down measurement, a decay-time measurement, a different acoustic path, a different gain or timing condition, or another ultrasonic operating condition selected to expose acoustic coupling outside the internal lumen.

In some embodiments, the bridge criterion may differ from a fluid-present criterion used for normal air-in-line detection. For example, the fluid-present criterion may evaluate acoustic coupling through the internal lumen under the sensing condition, while the bridge criterion may evaluate whether response data under the bridge-diagnostic condition indicate acoustic coupling along or around an exterior surface of the fluid conduit. When the bridge criterion is satisfied, the result may be treated as an alarmable unreliable-sensing condition because an air bubble may not be detected if the acoustic signal is being coupled around the tube instead of through the fluid path.

In some embodiments, the external acoustic bridge may include an acoustic coupling path external to the internal lumen of the fluid conduit. The external acoustic bridge may be caused by moisture, water, gel, coupling material, residue, cleaning solution, condensation, spilled fluid, medication, biological fluid, blood, adhesive, detergent, lubricious coating, tube residue, particulate matter, material trapped between the fluid conduit and a housing, material wicked into a channel, material spanning a transmitter-receiver gap, or another bridge-forming material outside the internal lumen.

In some embodiments, the controller may determine an external-bridge condition during an expected empty-conduit condition. The expected empty-conduit condition may be established from a door-open state, a door-close state following set loading, an absent-tubing state, an unprimed-tubing state, a flow-stop state, a set-loading state, a latch state, a priming state, a maintenance state, a commanded purge state, a user-commanded self-test state, a service command, or another state indicating that a reliable fluid-present state should not yet be asserted.

In some embodiments, the bridge criterion may include a bridge metric computed from one or more ultrasonic response features. The bridge metric may include an off-band magnitude, an off-band-to-operating-frequency ratio, a difference between an off-band magnitude and an expected empty response, an area under a frequency-response curve, a peak width, a slope, a phase value, a ring-down value, a decay-time value, a quality-factor value, a diagnostic-window value, a correlation score, a classifier score, an agreement score across multiple acoustic paths, a count of diagnostic conditions satisfying corresponding thresholds, or a combination thereof.

In some embodiments, memory may store calibration data for selecting or evaluating the first sensing condition, the second diagnostic condition, the bridge criterion, or other operating parameters. The calibration data may include per-unit calibration data, per-set calibration data, tubing-type calibration data, tube-material data, set-identification data, gain data, phase-offset data, response-profile data, expected empty-response data, expected primed-response data, expected bridged-response data, temperature-compensation data, pressure-compensation data, service-history data, or combinations thereof.

In some embodiments, the controller may obtain at least part of the calibration data from non-volatile memory, a barcode, a two-dimensional code, an RFID device, an optical identifier hole pattern, a keyed mechanical feature, a pump database, a service-tool entry, a remote configuration record, a disposable-set memory, or another source associated with a sensor assembly, pump, tube, cassette, flow-stop component, or disposable administration set.

In some embodiments, a calibration operation may select the bridge-diagnostic condition based on a response profile measured during manufacturing, service, installation, startup, loading of a disposable set, or another calibration operation. The bridge-diagnostic condition may be selected to provide greater separation between an externally bridged condition and an unbridged condition than a separation provided under the sensing condition. A diagnostic frequency may correspond to a local peak, local minimum, shoulder, slope region, phase-transition region, ring-down region, or other portion of a frequency response that may show greater sensitivity to an exterior-surface acoustic bridge than to liquid within the internal lumen. The selected bridge-diagnostic condition may be used to trigger an alarm or sensor-fault response when bridging would make normal air-in-line detection unreliable.

In some embodiments, the signal-processing circuit may generate in-phase signal data and quadrature signal data from a receiver signal. The controller may compute a magnitude M representing a distance between an instantaneous I/Q response vector and a reference I/Q response vector, for example according to M=√((I_{N}−-I_{P})²+(Q_{N}−Q_{P})²), where I_{N} is an instantaneous in-phase component, I_{P} is a baseline in-phase component, Q_{N} is an instantaneous quadrature component, and Q_{P} is a baseline quadrature component. Other magnitude, phase, ratio, or classifier features may additionally or alternatively be used for air/fluid discrimination and bridge diagnostics.

In some embodiments, the signal-processing circuit may include an active-rectification circuit configured to switch between a receiver signal and an inverted version of the receiver signal using first and second switching signals. The first and second switching signals may be approximately ninety degrees out of phase with each other. The active-rectification circuit may include first and second integrators and sample-and-hold circuits configured to sample primary response windows and diagnostic response windows. At least one diagnostic response window may be used to compensate for offset, identify saturation, identify drift, identify switch charge injection, identify an integrator reset fault, qualify a candidate fluid-present state, or identify an alarmable diagnostically invalid ultrasonic path.

In some embodiments, the fluid-line monitoring system may include a sensor housing configured to mechanically register the fluid conduit relative to the ultrasonic transducer arrangement. The sensor housing may include a conduit-receiving groove, a V-groove, a U-groove, an arcuate groove, a tube-registration surface, an alignment feature, a clamp, an epoxy-encapsulated transducer support, a drainage feature, a vent, a weep channel, a wicking break, a hydrophobic surface, an oleophobic surface, a low-surface-energy coating, an acoustic isolation gap, a relief channel, or another feature configured to position the conduit or reduce retained bridge-forming material along an exterior surface of the conduit.

In some embodiments, a pump may include a pump housing with a tube-receiving region configured to receive a tube, a pump mechanism configured to move fluid through the tube, a flow-stop interface configured to receive or actuate a flow-stop assembly associated with the tube, an ultrasonic air detector positioned relative to the tube, and a pump controller configured to perform one or more of the sensing, bridge-diagnostic, reliability-gating, bridge-alarm, air-detection, and pump-control operations described herein.

In some embodiments, the pump controller may select one or more bridge-diagnostic parameters based on a flow-stop identification signal, an optical identifier of a disposable set, a keyed geometry of the disposable set, a pump-door state, a latch state, a carriage state, a flow-stop state, a valve state, a pump-cycle state, a tube-loading state, a pressure signal, a temperature signal, or a combination thereof. The selected parameters may include a first sensing condition, a second diagnostic condition, one or more diagnostic frequencies, bridge thresholds, empty-response profiles, primed-response profiles, gain settings, pressure compensation, temperature compensation, and air-volume thresholds.

In some embodiments, the controller may respond to an external acoustic bridge by generating an alarm condition, generating a bridge alarm, generating an air-detection-unreliable alarm, generating a sensor-fault alarm, generating a maintenance prompt, generating a cleaning prompt, generating a reload prompt, inhibiting pumping, preventing therapy from starting, stopping infusion, storing a diagnostic log entry, requiring confirmation by another sensing modality before clearing the alarm condition, derating a confidence score, preventing an air-clear indication, modifying a threshold, or communicating with a user interface, service tool, hospital information system, or remote monitoring system.

In some embodiments, the controller may maintain a bridge state machine. The bridge state machine may include an untested state, a bridge-suspected state, a bridge-detected state, a bridge-cleared state, a diagnostically invalid state, and a sensor-disabled state. The controller may transition from a bridge-detected state to a bridge-cleared state only after a clearing test indicates that a bridge metric satisfies a clearing criterion. The clearing test may be performed after a cleaning prompt, a set reload, a purge sequence, a dwell period, a drying period, a pump-door opening and closing sequence, or another clearing operation.

In some embodiments, the ultrasonic transducer arrangement may include one transmitter and one receiver, one transmitter and multiple receivers, multiple transmitters and one receiver, multiple transmitters and multiple receivers, aligned transducer pairs, anti-aligned transducer pairs, upstream and downstream acoustic paths, angled acoustic paths, staggered acoustic paths, redundant acoustic paths, or combinations thereof. Agreement or disagreement among acoustic paths may be used to localize a bridge, distinguish external bridge material from in-lumen fluid, estimate bubble position, estimate bubble volume, determine bubble direction, or validate a bridge-clearing event.

In some embodiments, the pump controller may determine a bubble-volume estimate using an upstream acoustic path, a downstream acoustic path, delivered delta volumes accumulated while each acoustic path satisfies an air-reporting condition, and an estimate-selection rule based on a relationship between an upstream raw air estimate and a downstream raw air estimate. The pump controller may inhibit pumping, alarm, prompt maintenance, adjust a therapy-volume estimate, or modify a delivered-volume estimate in response to the bubble-volume estimate or the external acoustic bridge.

In some embodiments, the pump or fluid-line monitoring system may use pressure sensing, compliant bladders, impeders, restrictions, nucleation features, air traps, check valves, heaters, vents, hydrophobic membranes, pressure-maintaining structures, or pump-actuation routines to manage bubble formation, reduce outgassing, compensate ultrasonic thresholds, refine bubble-volume estimates, or improve delivered-volume estimates. Pump actuation may be interrupted, reversed, paused, or otherwise controlled to dislodge bubbles from the fluid line.

In some embodiments, any of the operations described herein may be performed by hardware, software, firmware, programmable logic, analog circuitry, digital circuitry, mixed-signal circuitry, one or more processors, one or more microcontrollers, one or more digital signal processors, one or more FPGAs, one or more CPLDs, one or more ASICs, or combinations thereof. Embodiments may include corresponding systems, apparatuses, methods, pump systems, fluid-line monitoring systems, medical sensor systems, controllers, computer programs, and non-transitory computer-readable storage media storing instructions that, when executed, cause one or more operations described herein to be performed.

The foregoing may introduce selected concepts in a simplified form that may be further described below in greater detail and may not limit the scope of any claim. Features described in connection with one embodiment may be combined with features described in connection with another embodiment unless such combination would be technically incompatible. Other systems, apparatuses, methods, features, and advantages may be apparent from the detailed description, drawings, and claims.

### BRIEF DESCRIPTION OF THE DRAWINGS

These and other aspects will become more apparent from the following detailed description of the various embodiments of the present disclosure with reference to the drawings wherein:

FIG. 1 shows a block diagram of a medical fluid delivery system including a fluid container, a pump, an air detector system, a fluid line, and a patient.

FIG. 2 shows a block diagram of an air detector system using active rectification.

FIG. 3 shows a block diagram of an ultrasonic sensing system including a transmitter, receiver, programmable oscillator, programmable logic, EEPROM calibration storage, quadrature sample-and-hold circuitry, low-pass buffering, an ADC, and a controller.

FIG. 4 shows a method of detecting air using active rectification.

FIG. 5 shows a circuit-level block diagram of in-phase and quadrature active rectification and magnitude detection.

FIG. 6 shows another method of detecting air using first and second switching networks and integrated outputs.

FIG. 7 shows a schematic representation of an active rectification circuit.

FIG. 8 shows a method of detecting air using positive-gain and negative-gain amplifier paths and switching between amplifier outputs.

FIGS. 9A and 9B show schematic portions of an active rectification circuit including signal generation, receive amplification, drive circuitry, switching, and filtering.

FIGS. 10-13 show representative signal traces illustrating drive, received, rectified, filtered, and quadrature signals under different conduit conditions.

FIG. 14A shows a frequency-response plot for different sensor states and an operating frequency selected during calibration.

FIGS. 14B and 14C show example bridging-test conditions and corresponding frequency responses.

FIG. 14D shows a frequency-sweep response for bridged and non-bridged test conditions.

FIGS. 15A-15C show schematic portions of an active rectification circuit including inverting and non-inverting amplifier paths, switching networks, integrators, sample-and-hold circuits, and output paths.

FIGS. 16A and 16B show schematic portions of control and power circuitry associated with the air detector circuit.

FIG. 17 shows comparator circuitry for high-threshold, low-threshold, and diagnostic comparisons.

FIGS. 18-21 show signal traces illustrating timing, integration, diagnostic sampling, sample-and-hold behavior, and air/fluid signal differences.

FIG. 22 shows a compensating reference-voltage circuit.

FIGS. 23A and 23B show representative signal traces for air and fluid conditions.

FIGS. 24A and 24B show schematic portions of another air-detection circuit including amplifier, switching, and control circuitry.

FIGS. 25A and 25B show schematic portions of control, power, oscillator, and transducer-interface circuitry.

FIGS. 26A-26C show schematic portions of circuitry configured to detect air in a fluid conduit.

FIGS. 27A-27C show schematic portions of a first detection circuit including amplifier, switching, integration, diagnostic, and comparator circuitry.

FIGS. 28A-28C show schematic portions of a second detection circuit including amplifier, switching, integration, diagnostic, and comparator circuitry.

FIG. 29 shows another ultrasonic sensing system including a transmitter, receiver, programmable oscillator, EEPROM calibration, programmable logic, quadrature sample-and-hold circuitry, low-pass buffering, and an MCU.

FIG. 30 shows a perspective view of a sensor connector or sensor assembly including a connector body, a flexible lead or mounting extension, and a latch or registration feature.

FIG. 31 shows a perspective view of a sensor clamp assembly including a mounting or alignment tab, a tube-receiving saddle or channel, a transducer support structure, a fastener or biasing member, and a base or cassette-interface structure.

FIG. 32 shows a perspective view of the sensor clamp assembly of FIG. 31 with internal conduit path and support features shown in dashed line.

FIG. 33 shows a cross-sectional view of a sensor clamp or conduit-registration structure including a fluid conduit, an upper clamp member, a lower conduit support, first and second ultrasonic elements or supports, and a controlled compression or contact region.

FIG. 34 shows a conduit-receiving region, groove, or channel geometry including a sidewall, bottom support, and relief, drainage, vent, hydrophobic, or cleaning feature.

FIG. 35 shows a method for dislodging bubbles in a fluid line by interrupting or reversing pump actuation.

FIG. 36 shows a method for detecting a bubble using multiple ultrasonic thresholds, trigger-threshold adjustment, and timers.

FIG. 37A shows an ultrasonic bubble sensor including one transmitter and two receivers.

FIG. 37B shows an ultrasonic bubble sensor including two transmitters and one receiver.

FIG. 37C shows an ultrasonic bubble sensor including two transmitters and two receivers in an aligned arrangement.

FIG. 38 shows a method for estimating air pumped downstream using upstream and downstream air detections.

FIG. 39 shows a method for updating raw upstream and downstream air estimates and fluid-clear volumes.

FIG. 40 shows a method for completing a bubble estimate using a relationship between upstream and downstream raw air estimates.

FIGS. 41A-41F show an in-line pressure sensor and clip configured to sense pressure-related deformation of a fluid line.

FIG. 42 shows another in-line pressure sensor including an insert positioned in a fluid line.

FIG. 43 shows a downstream bladder or compliant tube section configured to smooth pulsatile flow or manage pressure.

FIG. 44 shows a tube section including impeders or nucleation features configured to affect bubble formation or pressure.

FIG. 45 shows another downstream bladder or compliant chamber section.

FIG. 46 shows a tubing section including nucleation sites and an air trap.

FIG. 47 shows a system including a check valve configured to maintain line pressure and reduce outgassing.

FIG. 48 shows a method for pumping fluid and adjusting a delivered-fluid estimate to account for air or bubbles.

### DETAILED DESCRIPTION

Exemplary features described with respect to one embodiment may be combined with features described with respect to another embodiment unless such combination is technically incompatible. The disclosure is not limited to any particular pump type, tube size, fluid type, signal polarity convention, circuit topology, or numerical frequency range unless expressly recited in a claim. The described embodiments are illustrative and non-limiting. Features described in connection with one embodiment may be combined with features described in connection with another embodiment unless such combination is technically incompatible. The words “about,” “approximately,” and similar terms encompass ordinary engineering tolerances and measurement variation. The terms “first,” “second,” and similar terms are used to distinguish elements and do not require a particular order unless expressly stated. The term “comprising” does not exclude additional elements or steps.

As used herein, a “fluid conduit” may include IV tubing, pump tubing, disposable set tubing, a cassette channel, a molded conduit, a bladder, a chamber, or another passage configured to carry medical fluid. The conduit may be flexible, semi-rigid, or rigid. The conduit may be part of a disposable administration set, cassette, flow stop, pump segment, reusable cartridge, or other medical fluid component.

As used herein, an “external bridging condition” refers to an acoustic coupling condition in which material outside an intended internal fluid path provides an ultrasonic coupling path that is not representative of liquid within the conduit. External bridging material may include, without limitation, water, gel, coupling material, cleaning solution, residue, condensation, spilled fluid, biological fluid, adhesive, or material trapped between a conduit and a housing. The bridge may occur around the conduit, through a clamp, through a housing surface, across a sensor channel, or through another external acoustic path.

As used herein, an “operating frequency” or “calibrated drive frequency” refers to a frequency selected for normal air/fluid discrimination. The selected frequency may be a resonance frequency, a near-resonance frequency, a frequency that maximizes fluid-to-air separation, a frequency that minimizes sensor variation, or a frequency selected by another calibration rule. An “off-band frequency” refers to a frequency different from the calibrated drive frequency and used for diagnostics, including bridge detection. The off-band frequency need not be outside every acoustic resonance of the system; it may be outside the selected normal operating band or otherwise selected to expose bridge-sensitive behavior.

As used herein, to invalidate or qualify a fluid-present determination may include preventing a fluid-present state from being asserted as a valid air-clear or fluid-present condition, derating a confidence score, increasing a threshold, requiring confirmation by another frequency or sensor, disregarding a measurement, generating a fault, generating an alarm condition, stopping or inhibiting a pump, requiring cleaning or reloading, recording a diagnostic condition, or presenting a warning to a user. Invalidation or qualification of a fluid-present determination is not a silencing of an air-in-line alarm. Rather, a bridge-detected state may itself be an alarmable condition because the ultrasonic signal may be going around the tubing rather than through the fluid path.

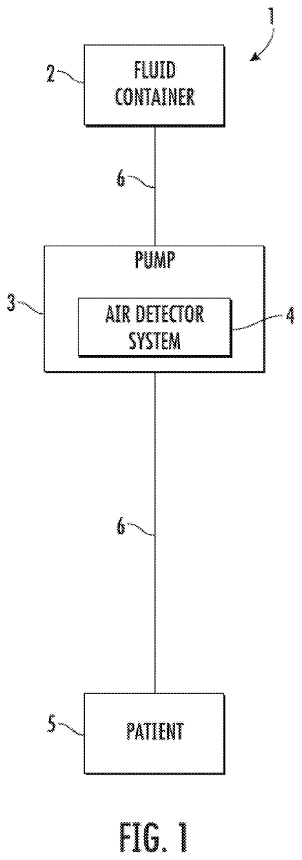

FIG. 1 illustrates a system 1 for infusing fluid into a patient 5. A fluid container 2 is coupled to a fluid line 6. A pump 3 may drive, regulate, monitor, or otherwise control fluid flow through the fluid line 6 toward the patient 5. The pump 3 includes or is coupled to an air detector system 4. Although a pump 3 is shown, the air detector system 4 may also be used with a gravity-fed system, a pressure-assisted system, a syringe pump, a peristaltic pump, a modular pump, a dialysis device, or another medical fluid-delivery device.

The air detector system 4 may detect, estimate, track, or categorize air in the fluid line 6. In some embodiments, the pump 3 modifies, adjusts, or accounts for air delivered to the patient as determined by the air detector system 4. For example, the pump 3 may issue an alarm or alert when the total amount of air detected during a therapy session exceeds a first threshold, when the amount of air detected during a predetermined period exceeds a second threshold, or when the amount of air detected during a most-recent rolling time period exceeds a third threshold. The thresholds may be the same or different. Air may be tracked by volume, mass, event count, event duration, or a combination thereof. Temperature, pressure, flow rate, fluid type, or tubing type may be used to refine the estimate.

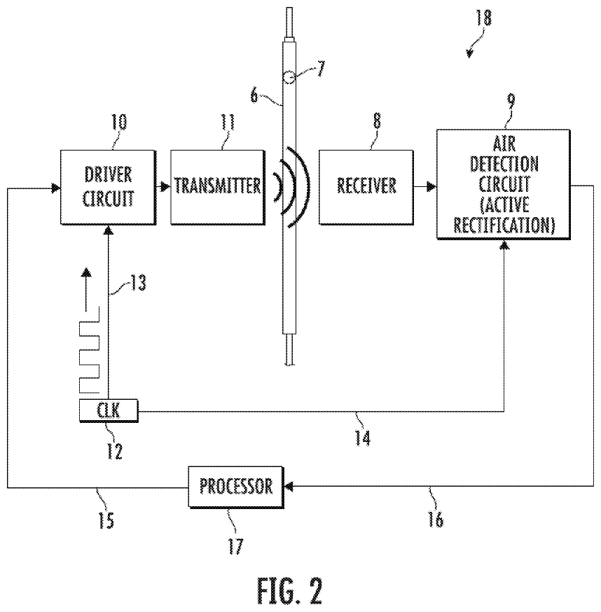

FIG. 2 shows an air detector system 18. The system 18 includes a driver circuit 10, an ultrasonic transmitter 11, an ultrasonic receiver 8, an air detection circuit 9, a clock 12, and a processor 17. The transmitter 11 and receiver 8 may be piezoelectric elements positioned on opposite sides of the fluid line 6. The processor 17 may signal the driver circuit 10 to drive the transmitter 11. The transmitter 11 generates ultrasonic energy that propagates through the fluid line 6 and any material within or adjacent to the fluid line 6. The receiver 8 transduces received ultrasonic energy into an electrical receiver signal.

The air detection circuit 9 receives the receiver signal and provides a processed signal to the processor 17. The processed signal may correspond to the amount of ultrasonic energy transmitted across the fluid line 6. In some embodiments, liquid in the fluid line 6 transmits more ultrasonic energy than air in the fluid line 6, so a higher processed magnitude indicates fluid and a lower processed magnitude indicates air. In other embodiments, signal polarity, inversion, filtering, or circuit convention may produce a different threshold relationship; accordingly, the controller may use any appropriate comparison, mapping, lookup table, classifier, or calibration model to determine fluid or air.

The air detection circuit 9 may perform active rectification, including synchronous rectification. Active rectification may be performed by switching between a receiver signal and an inverted receiver signal using one or more switching signals synchronized to a transmitted waveform, a received waveform, or a derived clock. Active rectification may improve detection of low-level ultrasonic signals while rejecting noise, phase ambiguity, or offset errors.

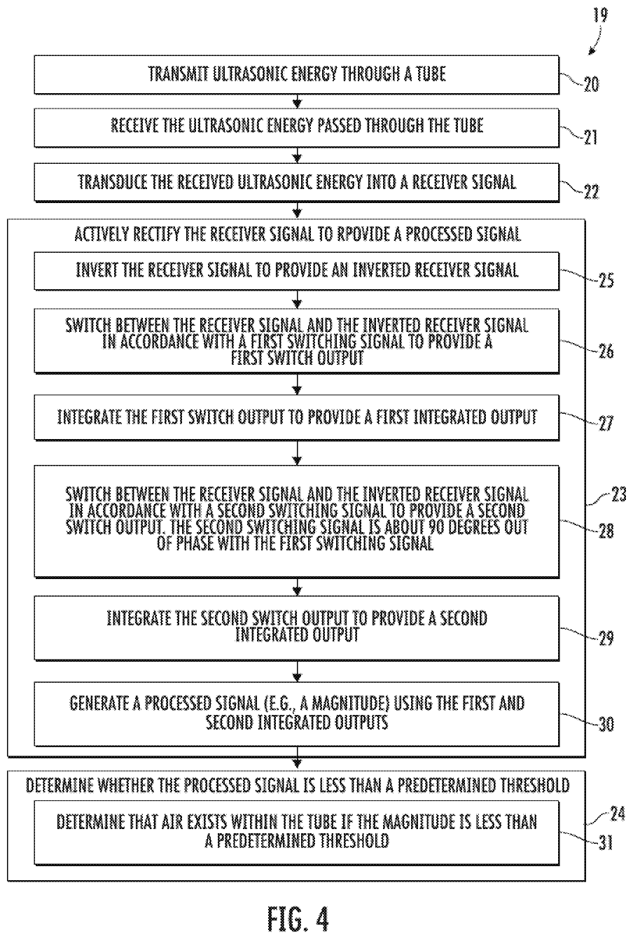

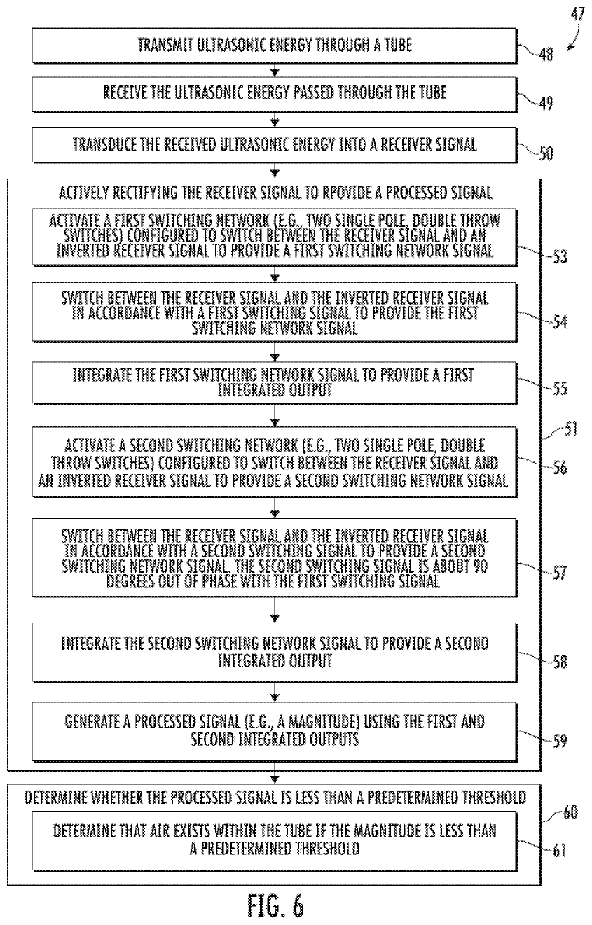

FIGS. 4 and 6 illustrate methods of detecting air using active rectification. In one method, ultrasonic energy is transmitted through a tube, ultrasonic energy passing through the tube is received, the received ultrasonic energy is transduced into a receiver signal, and the receiver signal is actively rectified to provide a processed signal. The receiver signal may be inverted to provide an inverted receiver signal. A first switching network may switch between the receiver signal and the inverted receiver signal in accordance with a first switching signal to provide a first switching output, and a second switching network may switch between the receiver signal and the inverted receiver signal in accordance with a second switching signal to provide a second switching output. The second switching signal may be approximately ninety degrees out of phase with the first switching signal. The first and second switching outputs may be integrated or filtered to provide first and second integrated outputs. A magnitude, vector magnitude, or other processed signal may be generated from the first and second integrated outputs and compared with one or more thresholds to determine whether air exists within the tube.

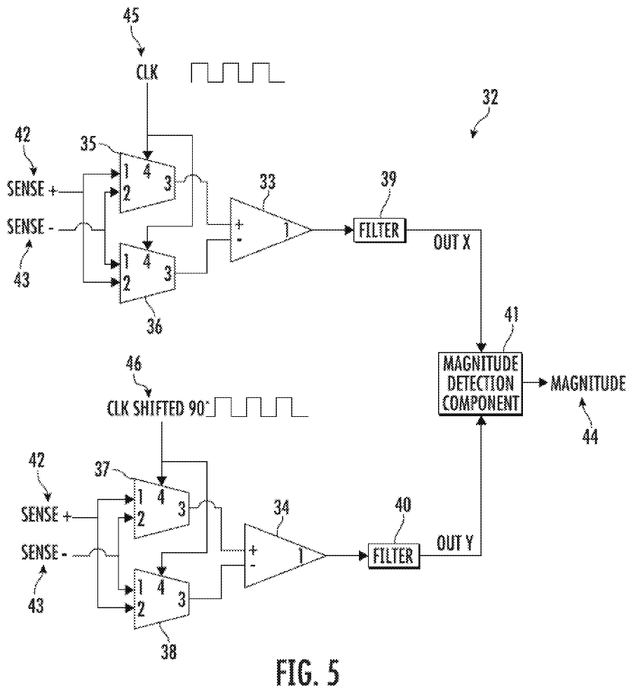

FIG. 5 illustrates an active-rectification circuit 32. A first switching path includes switches 35 and 36, amplifier 33, filter 39, and an output OUT X. A second switching path includes switches 37 and 38, amplifier 34, filter 40, and an output OUT Y. The first path is driven by a clock 45, and the second path is driven by a clock 46 shifted approximately ninety degrees relative to the first clock. A magnitude detection component 41 receives OUT X and OUT Y and generates a magnitude 44. In some embodiments, OUT X and OUT Y correspond to in-phase and quadrature components of the received signal.

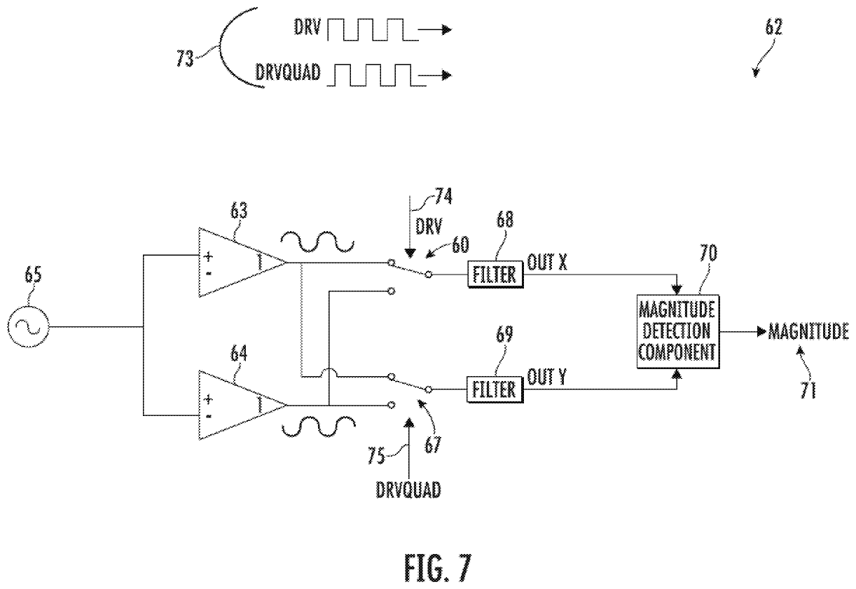

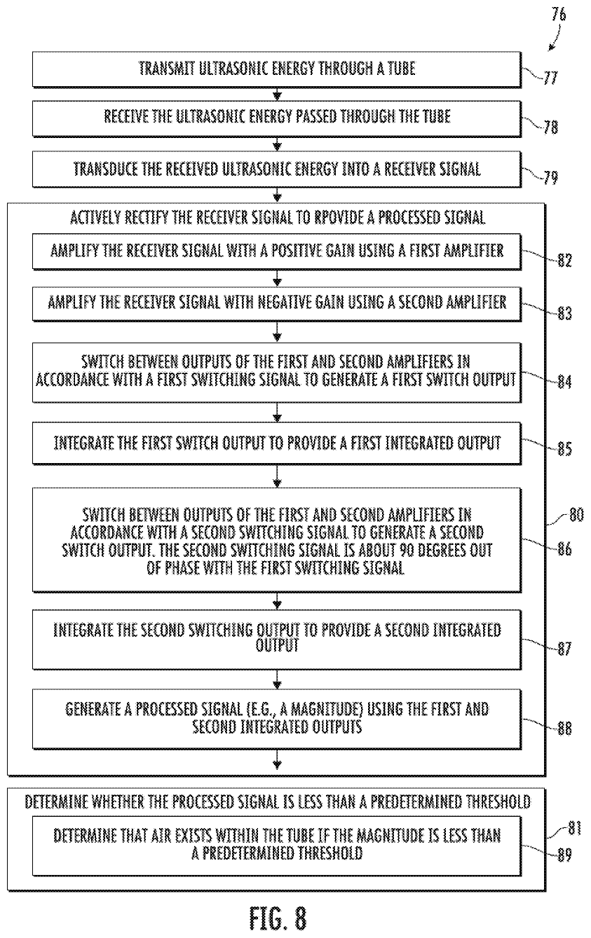

FIG. 7 illustrates another circuit 62 in which first and second amplifier paths 63 and 64 drive first and second filters 68 and 69 through switches 74 and 75. A magnitude detection component 70 generates a magnitude 71. FIG. 8 illustrates a method in which the receiver signal is amplified with a positive gain using a first amplifier and with a negative gain using a second amplifier. A switching network switches between outputs of the amplifiers in accordance with a switching signal, and integrated outputs are used to generate a processed signal.

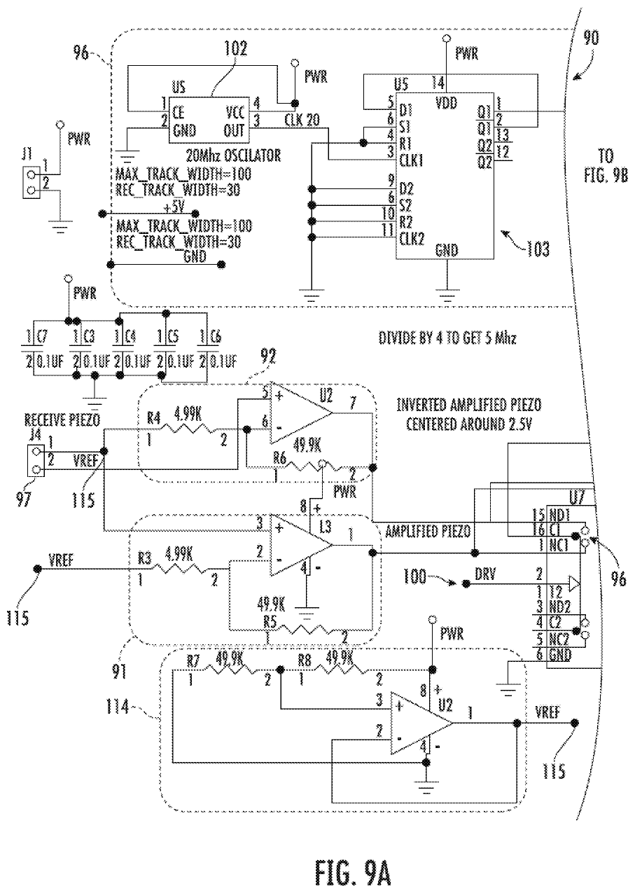

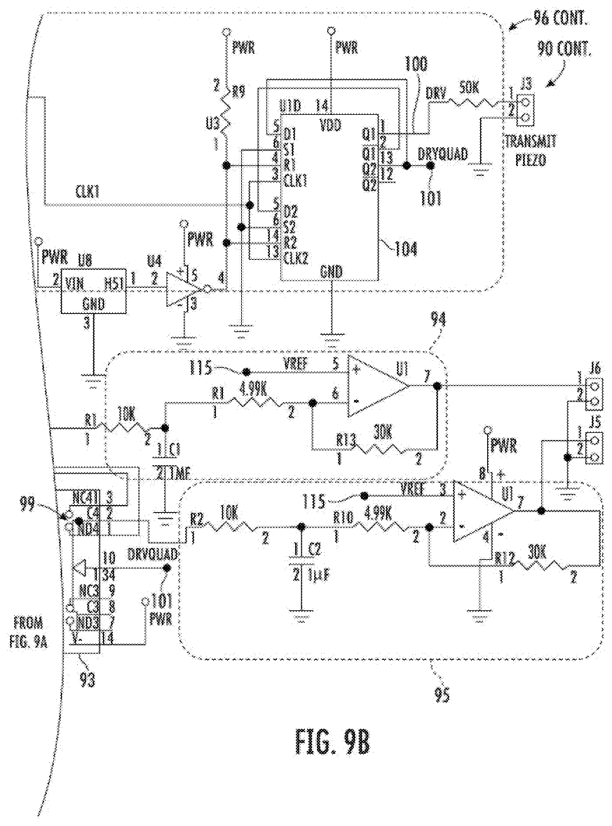

FIGS. 9A and 9B show an example circuit 90. A received piezoelectric signal may be provided to inverting and non-inverting amplification paths. A reference voltage may bias the receiver signal and amplifier outputs. A signal generating circuit may generate switching signals used for transmitter drive and synchronous rectification. The switching signals may be derived from a reference clock, divider, flip-flops, programmable logic, or other timing circuit. The circuit may include filters for producing in-phase and quadrature outputs used by a controller to calculate a magnitude.

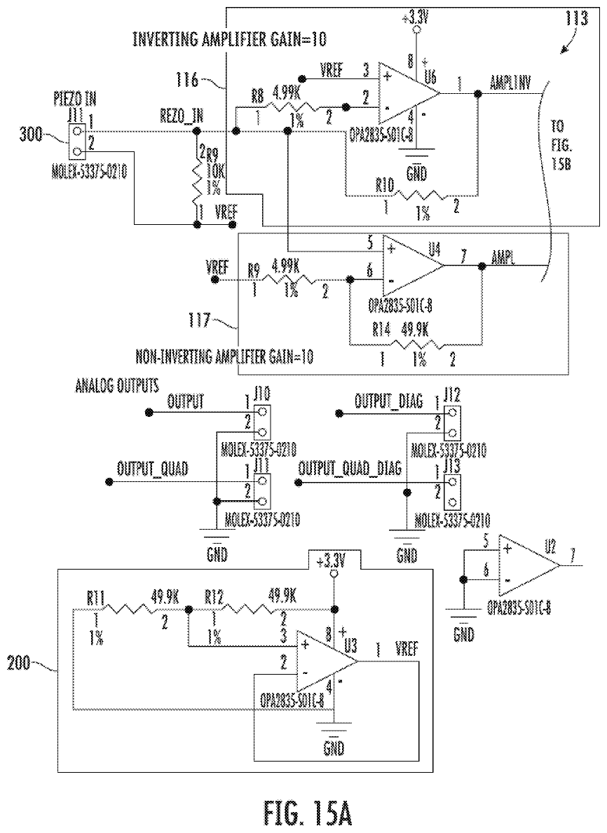

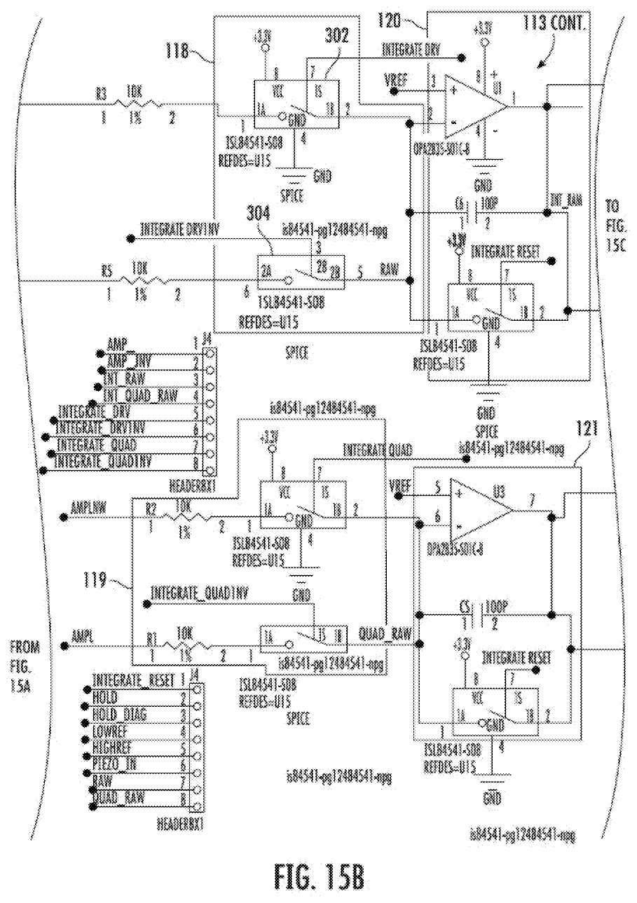

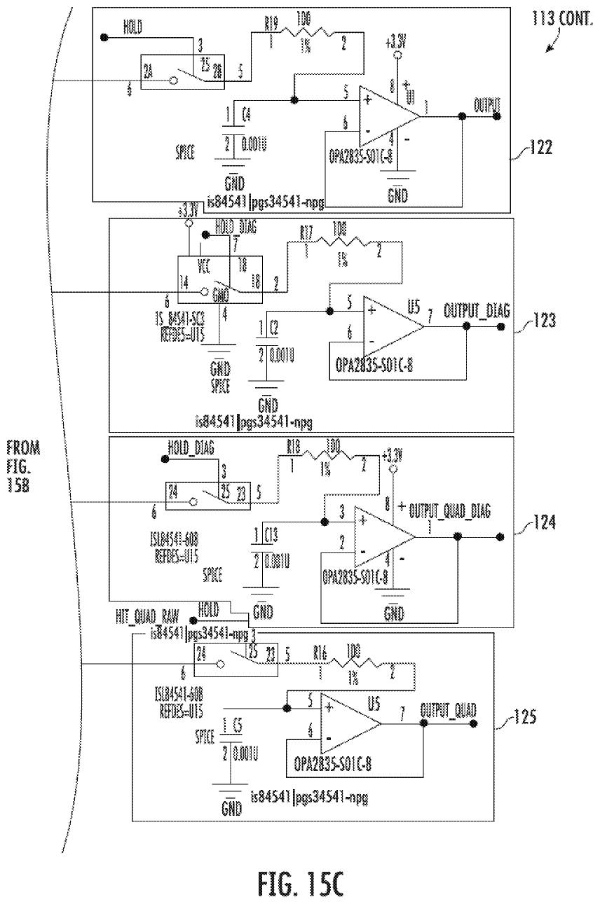

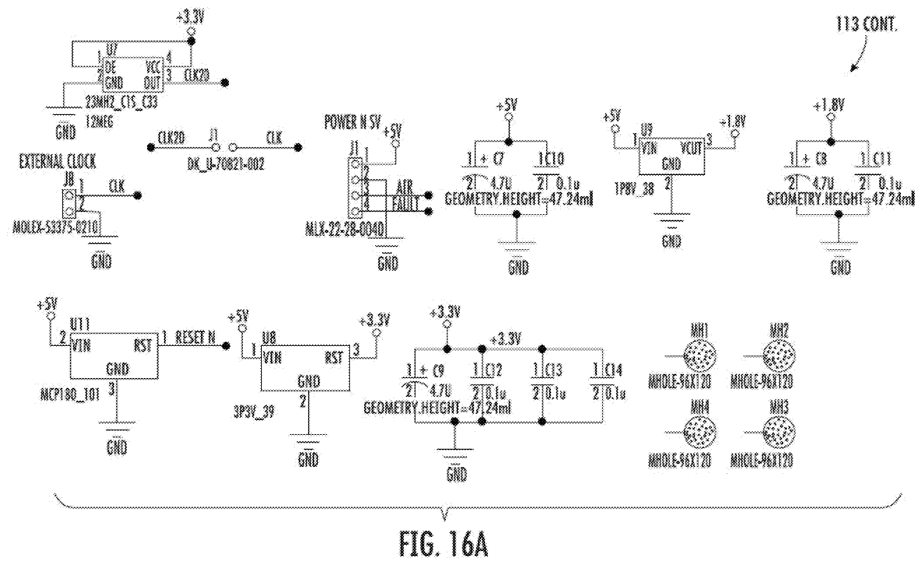

FIGS. 15A-15C illustrate a circuit 113. A receiver signal is received through a connection 300. The circuit 113 includes an inverting amplifier 116 and a non-inverting amplifier 117. The amplifier outputs are provided to first and second switching networks 118 and 119. The first switching network 118 switches between amplified and inverted-amplified receiver signals in accordance with a first switching signal. The second switching network 119 switches between amplified and inverted-amplified receiver signals in accordance with a quadrature switching signal. The first switching network output is provided to a first integrator 120, and the second switching network output is provided to a second integrator 121. The integrated outputs may be sampled by sample-and-hold circuits 122, 123, 124, and 125.

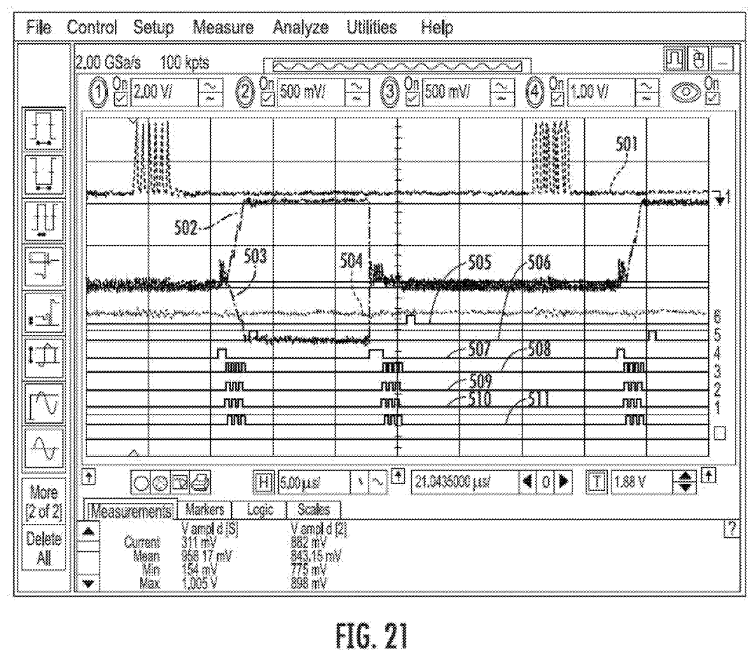

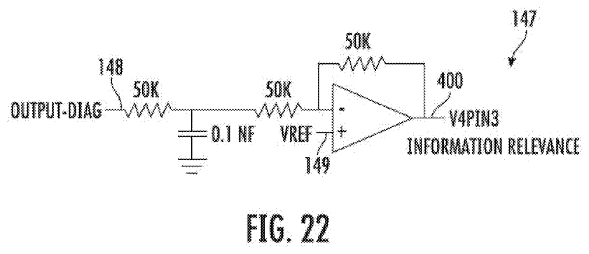

In some embodiments, one sample-and-hold circuit samples a signal window corresponding to the primary ultrasonic response and another sample-and-hold circuit samples a diagnostic window after the acoustic response has substantially dissipated. The diagnostic sample may represent offset, drift, amplifier error, switching artifact, charge injection, integrator reset error, or another baseline condition. The controller may subtract the diagnostic sample from the primary sample, use the diagnostic sample to generate a compensating reference voltage, or compare the diagnostic sample with one or more diagnostic thresholds. FIG. 22 illustrates a compensating reference-voltage circuit 147 that receives an output diagnostic signal 148 and provides a reference-related output 400.

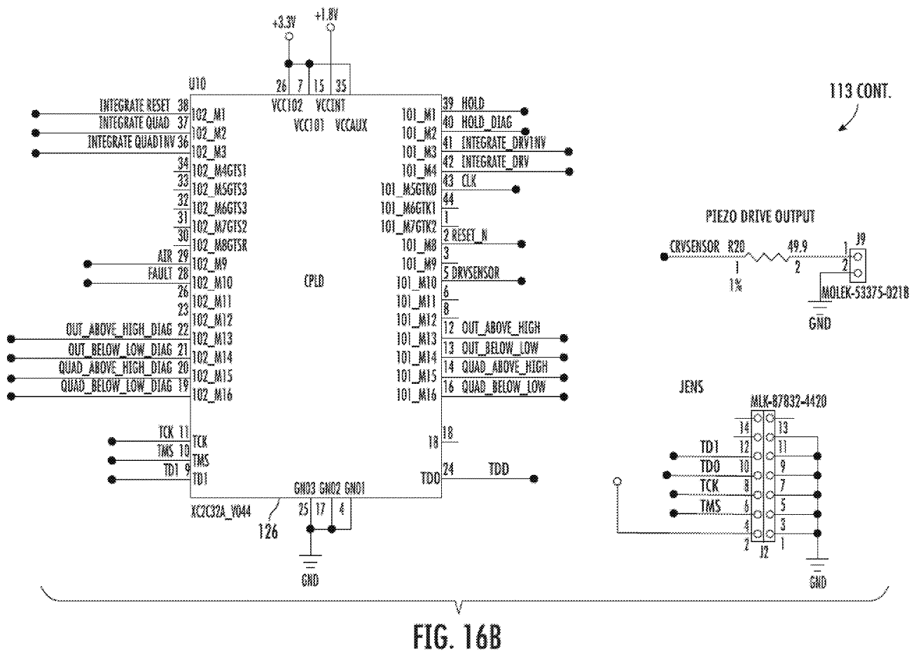

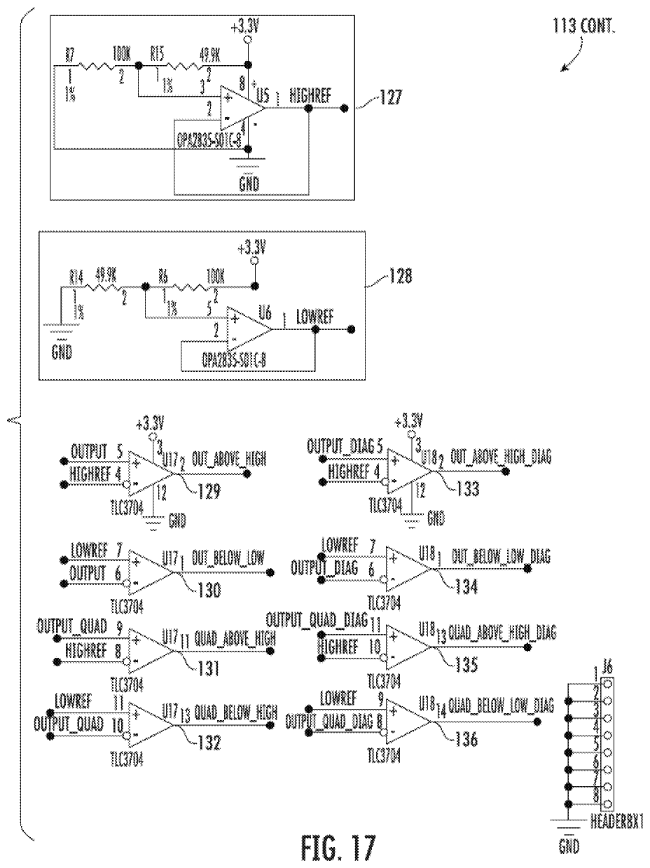

FIG. 17 illustrates comparators that compare primary and diagnostic outputs against high and low thresholds. In some embodiments, comparator outputs associated with the primary in-phase and quadrature channels indicate whether fluid is present. Comparator outputs associated with diagnostic channels may indicate a bridge condition, fault condition, offset, saturation, unexpected drift, or another malfunction. When one or more primary comparator outputs meet a fluid condition and the diagnostic outputs do not indicate bridging, the controller may determine that fluid is present. When the primary comparator outputs do not meet a fluid condition, the controller may determine that air is present. When diagnostic comparator results indicate an external bridge, the controller may invalidate the fluid/air determination and generate an alarm or sensor-fault condition because air detection through the tube may be unreliable.

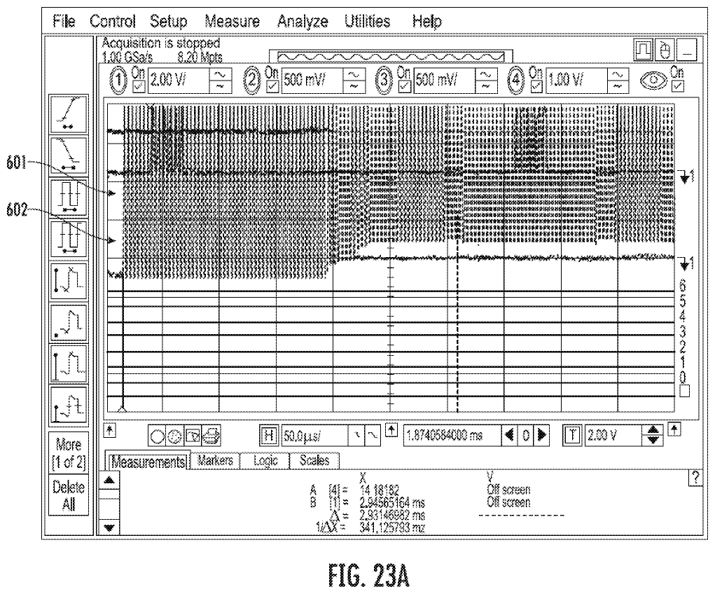

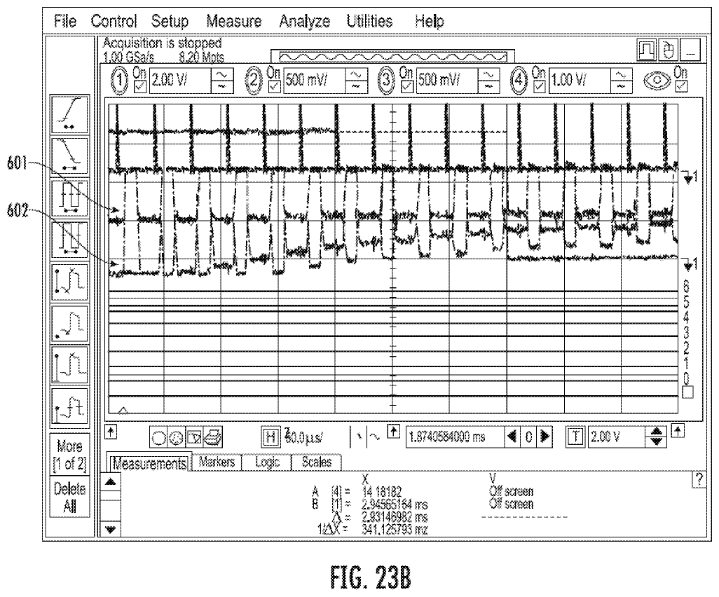

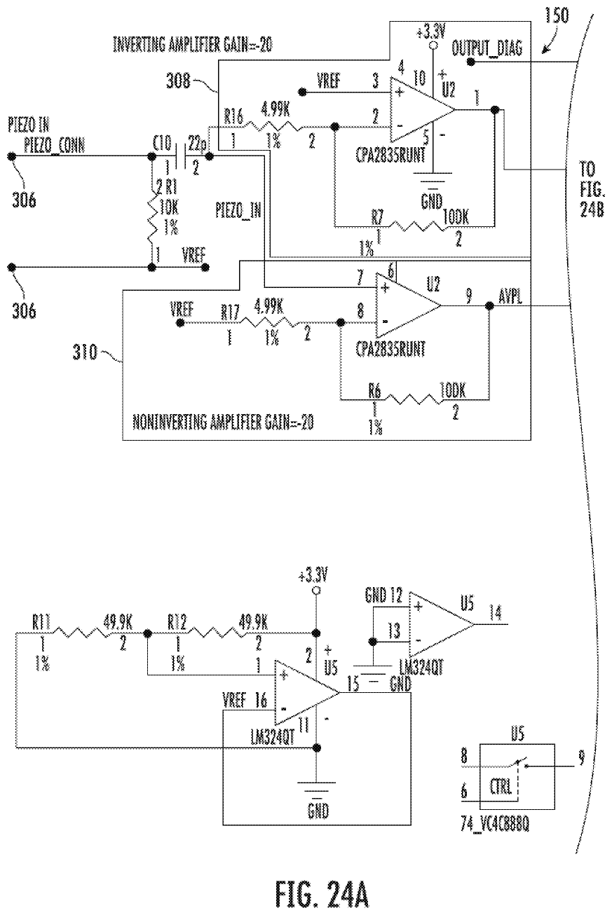

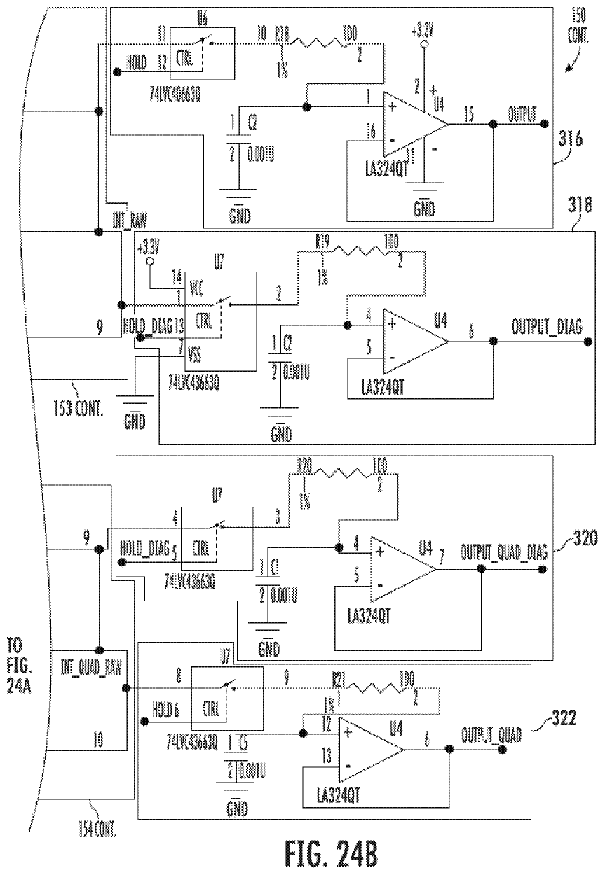

FIGS. 24A-24B show another circuit 150. The circuit 150 includes an inverting amplifier 308, a non-inverting amplifier 310, switching networks 312 and 314, integrators 153 and 154, and sample-and-hold circuits 316, 318, 320, and 322. A programmable logic device, CPLD, FPGA, microcontroller, or other controller may generate first and second switching signals, control reset of the integrators, control sample-and-hold timing, receive or digitize output signals, and issue alarms, alerts, or pump-control signals based on detected air or detected bridge conditions.

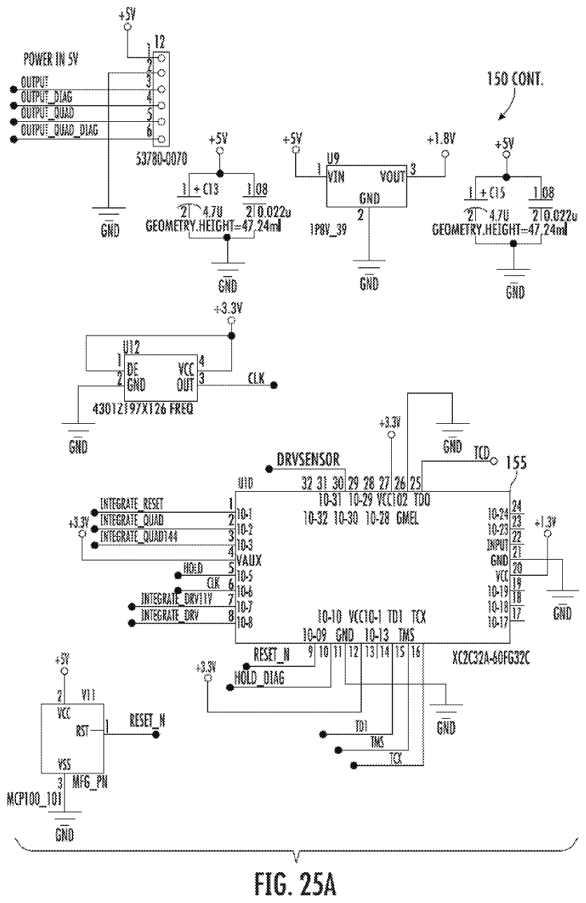

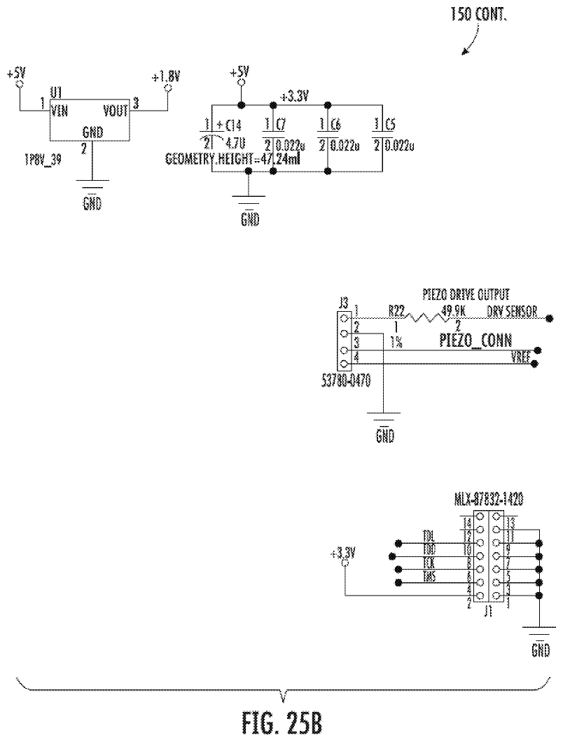

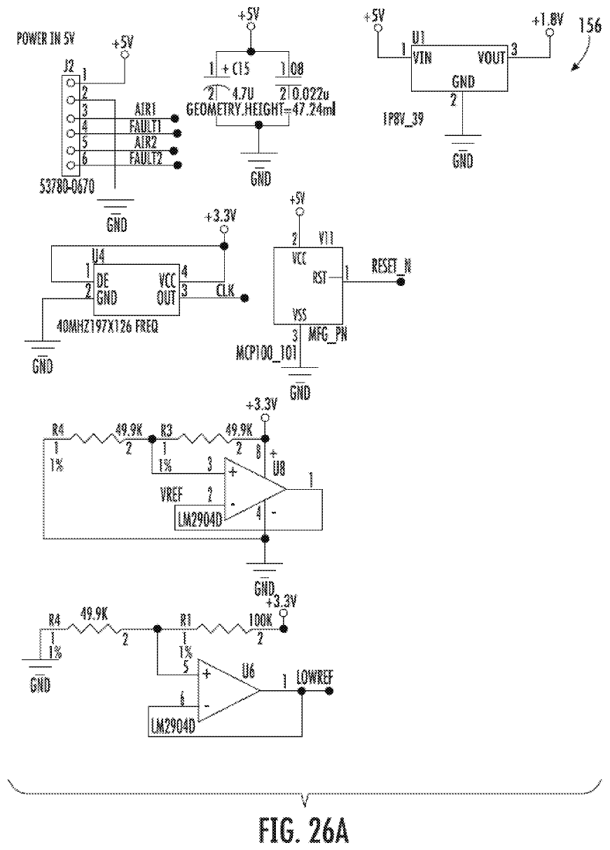

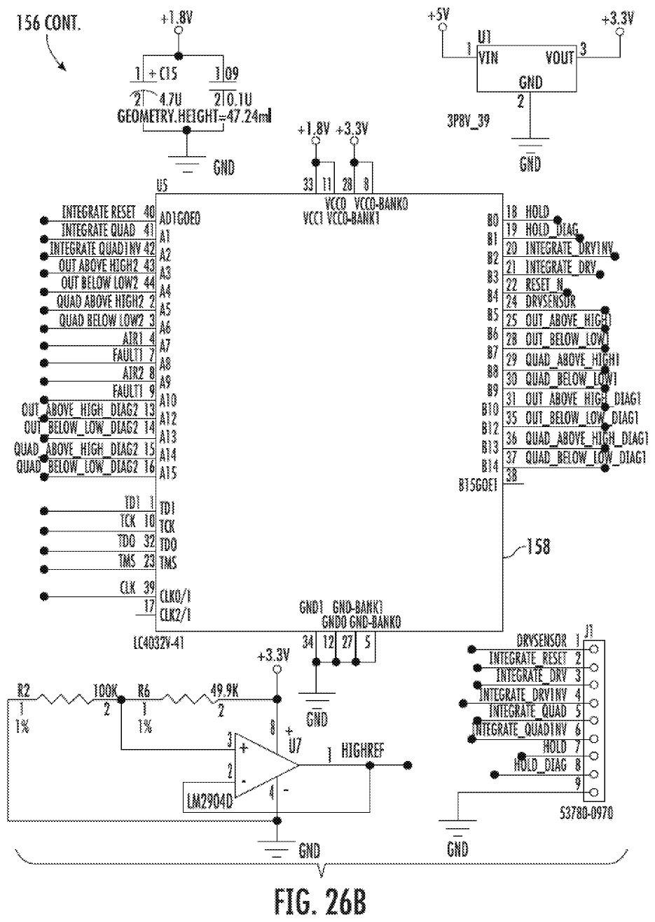

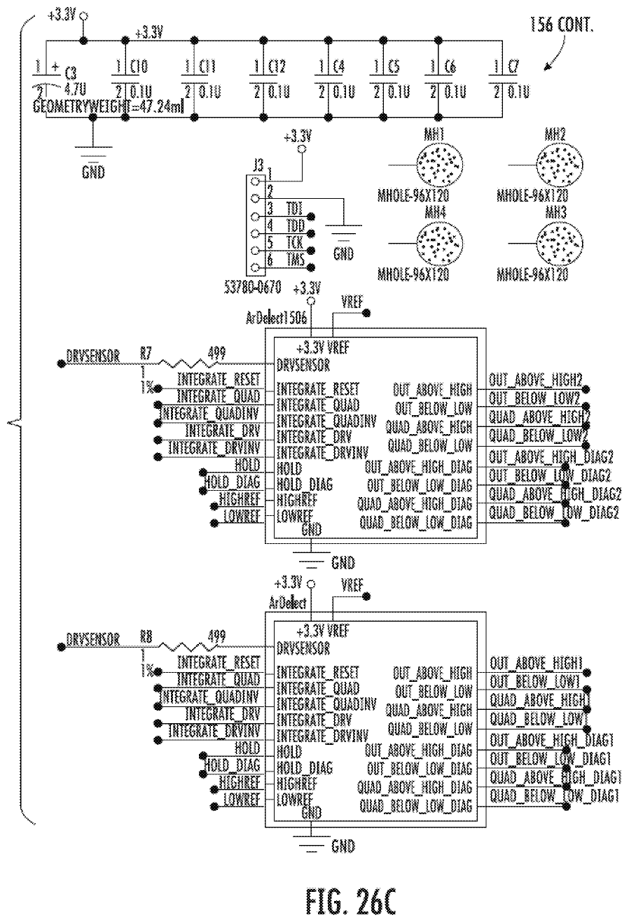

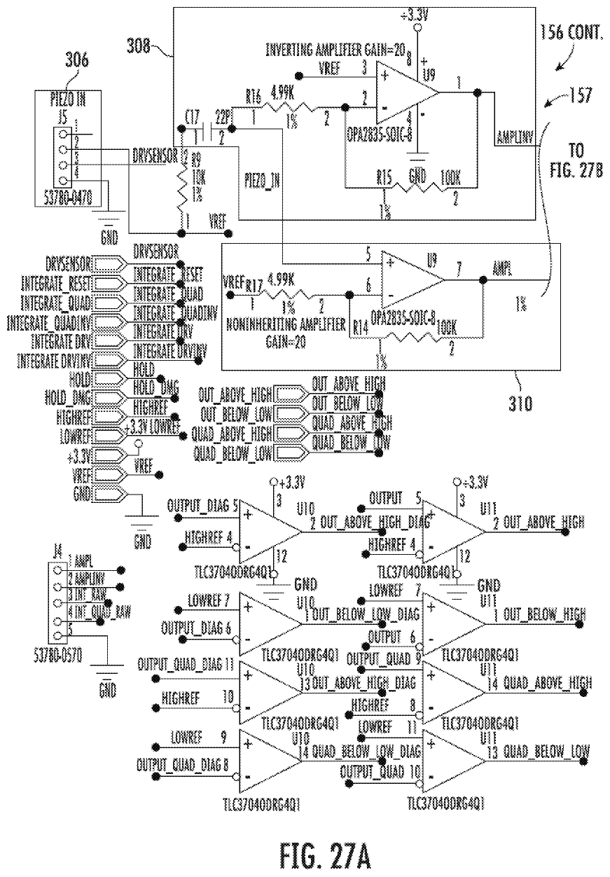

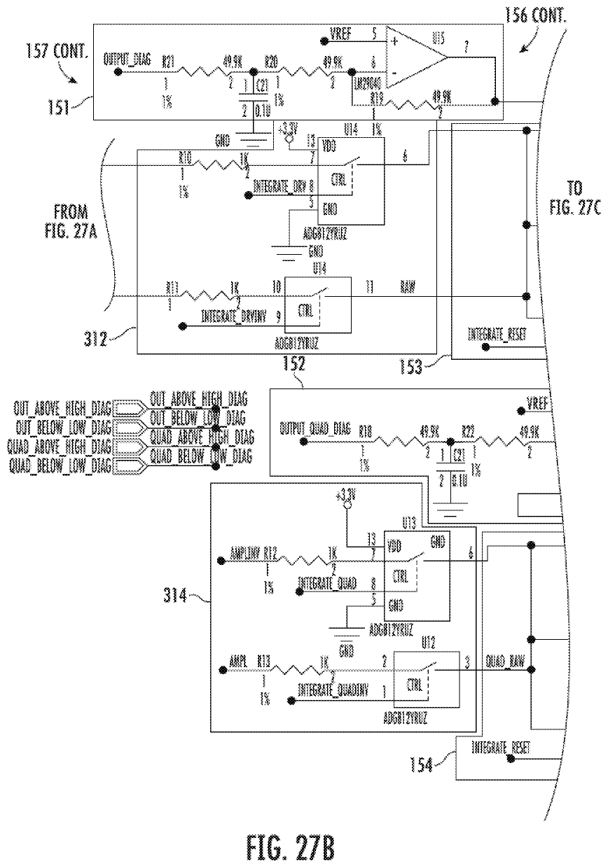

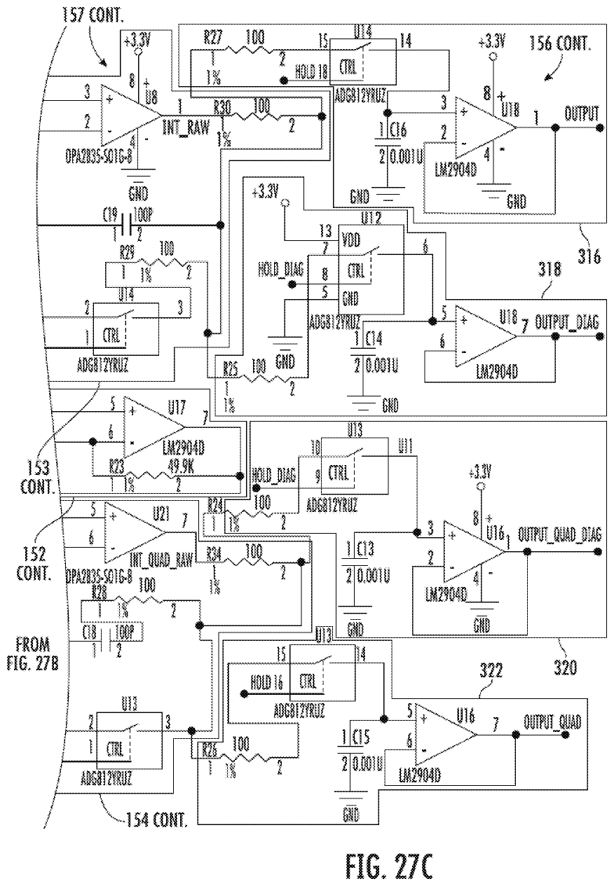

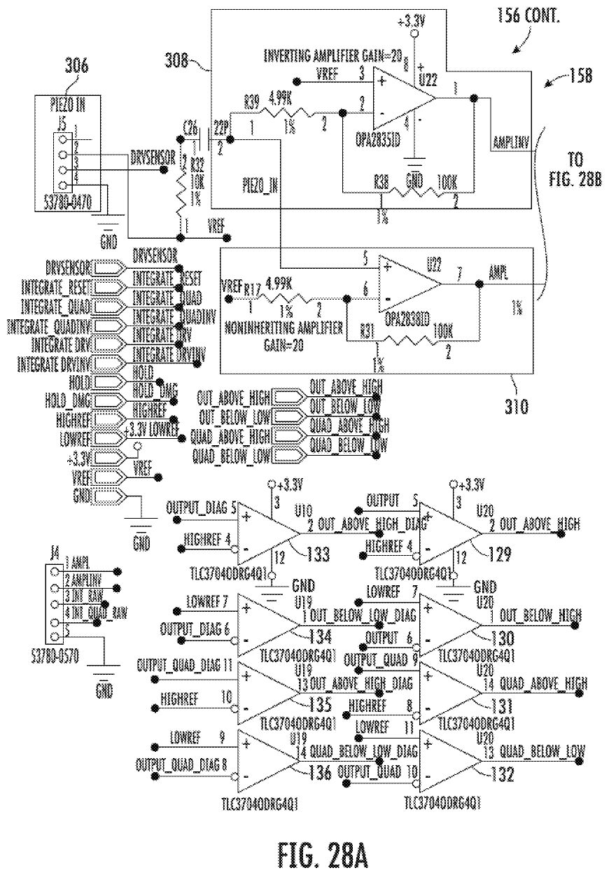

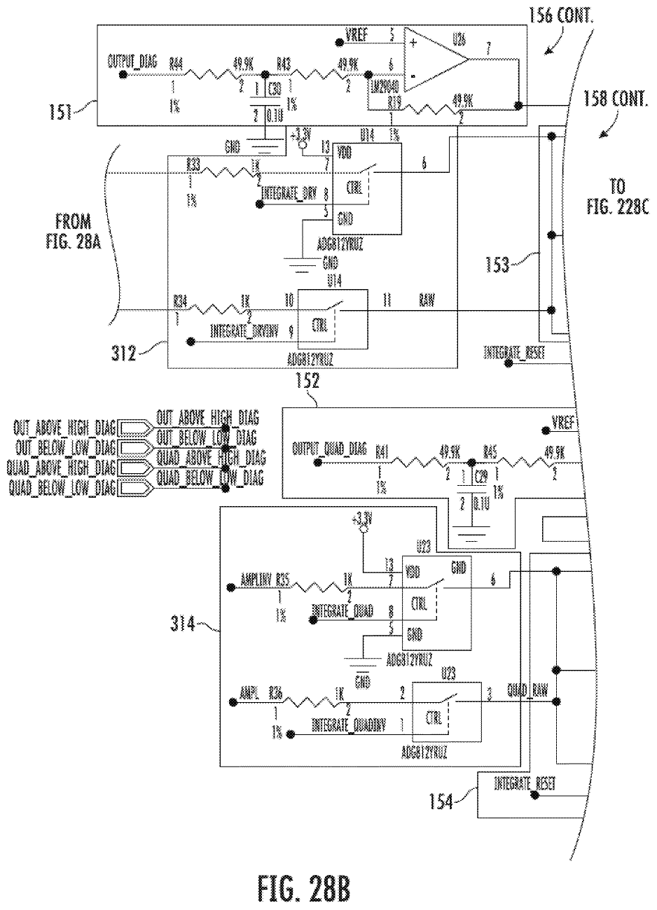

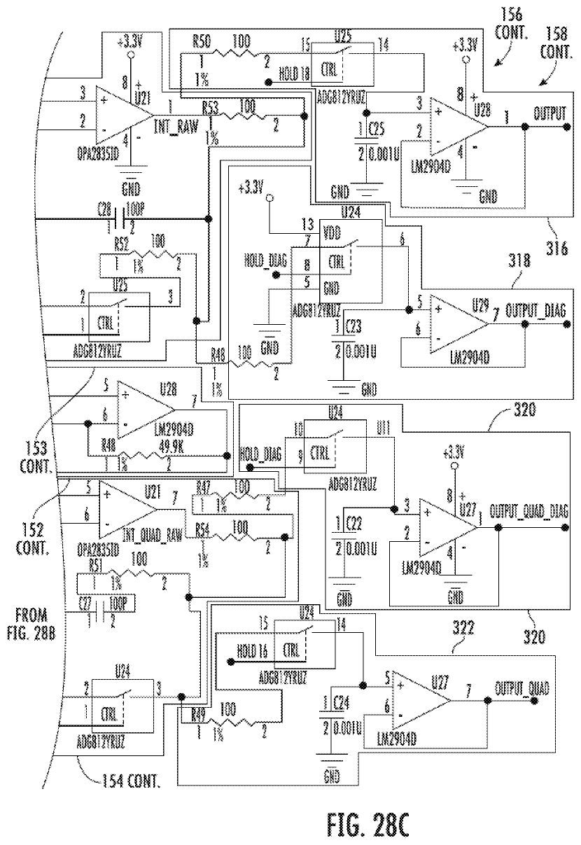

FIGS. 26A-28C illustrate circuitry for detecting air in one or more fluid conduits. In some embodiments, a circuit includes a first detection channel for a first tube and a second detection channel for a second tube. The channels may share a controller, oscillator, programmable logic device, threshold generator, power circuit, or diagnostic circuit. In other embodiments, the channels operate independently or are multiplexed to reduce component count.

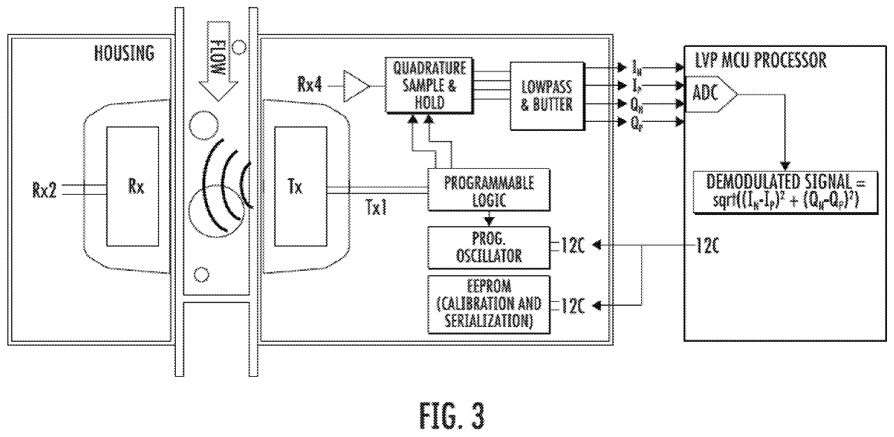

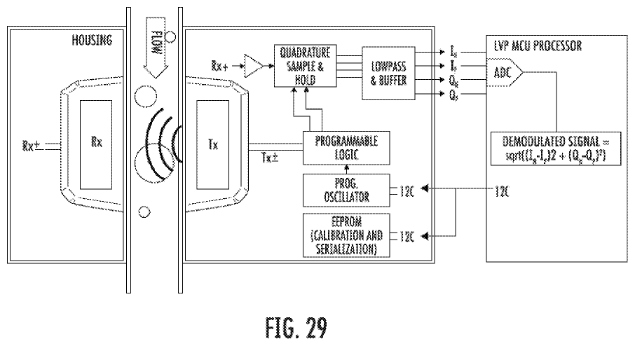

FIG. 3 and FIG. 29 illustrate ultrasonic sensing systems configured for air-in-line detection, fluid presence detection, bridge detection, and air-volume estimation. A transmitter Tx and a receiver Rx are positioned on opposite sides of a fluid conduit. The transmitter and receiver may be housed in an epoxy-encapsulated structure, mounted in a clamp, disposed in a pump housing, or otherwise mechanically registered with the conduit. The sensing system may include an EEPROM or other non-volatile memory, a programmable oscillator, a programmable logic unit, a quadrature sample-and-hold stage, a low-pass and buffer stage, an ADC, and a controller such as a low-power microcontroller.

During operation, the programmable oscillator drives the transmitter at a selected drive frequency. The receiver generates a receiver signal that is provided to a quadrature sample-and-hold stage. The quadrature sample-and-hold stage generates an in-phase component and a quadrature component of the receiver signal. In some embodiments, the in-phase component includes an instantaneous in-phase value I_{N} and a corresponding reference, filtered, averaged, low-pass, stored, or baseline in-phase value I_{P}. The quadrature component includes an instantaneous quadrature value Q_{N} and a corresponding reference, filtered, averaged, low-pass, stored, or baseline quadrature value Q_{P}. The controller may compute a demodulated magnitude M representing a distance between an instantaneous I/Q response vector and a reference I/Q response vector, for example according to: M=√((I_{N}−I_{P})²+(Q_{N}−Q_{P})²).

The magnitude M may be compared to one or more thresholds to determine whether fluid is present, whether air is present, whether a bubble has passed, whether the tube is empty, whether the conduit is primed, whether the conduit is absent, or whether a fault condition exists. The controller may also apply hysteresis, debounce logic, rolling windows, adaptive thresholds, temperature compensation, pressure compensation, fluid-type compensation, tubing-type compensation, bridge compensation, offset compensation, drift compensation, gain compensation, or combinations thereof.

The reference, filtered, averaged, low-pass, stored, or baseline values I_{P} and Q_{P} may be generated by analog filters, digital filters, sample-and-hold stages, moving averages, exponential filters, calibration data, diagnostic sample windows, expected-state measurements, or other baseline-estimation techniques. In some embodiments, I_{N} and Q_{N} are instantaneous values captured during an acoustic-response window, while I_{P} and Q_{P} are reference values captured during a different window, generated from prior samples, generated during a calibration or diagnostic routine, or retrieved from memory. In other embodiments, I_{P} and Q_{P} are low-pass filtered versions of the instantaneous in-phase and quadrature channels.

The acoustic response of an ultrasonic sensor may depend on transducer characteristics, tubing material, conduit geometry, sensor clamping force, epoxy encapsulation, environmental conditions, and manufacturing tolerances. Accordingly, in some embodiments each sensor is calibrated during manufacturing, service, installation, startup, or loading of a disposable set. A calibration routine may sweep a frequency band and measure a response magnitude at each test frequency. A calibrated drive frequency may be selected based on peak magnitude, desired separation between air and fluid states, robustness to tubing variation, minimized sensitivity to external bridging, or another response-shape criterion.

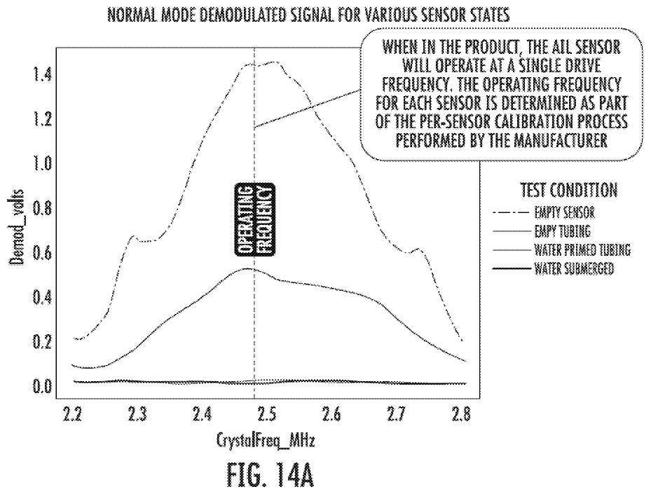

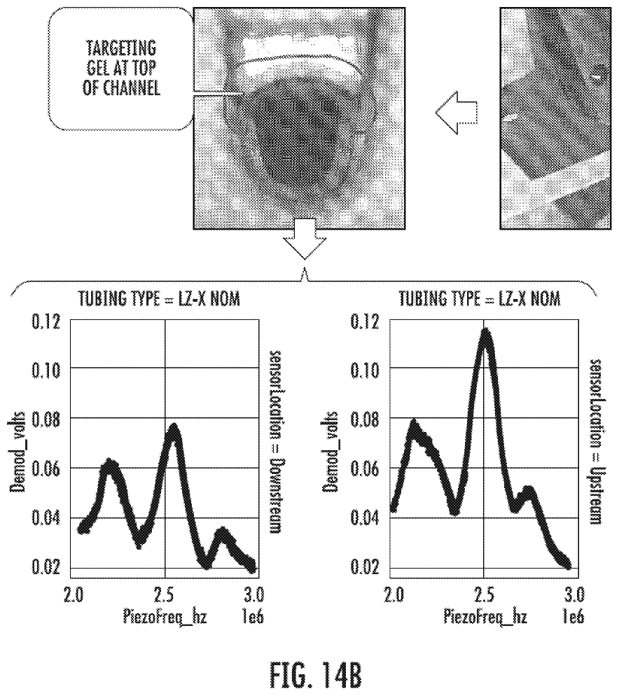

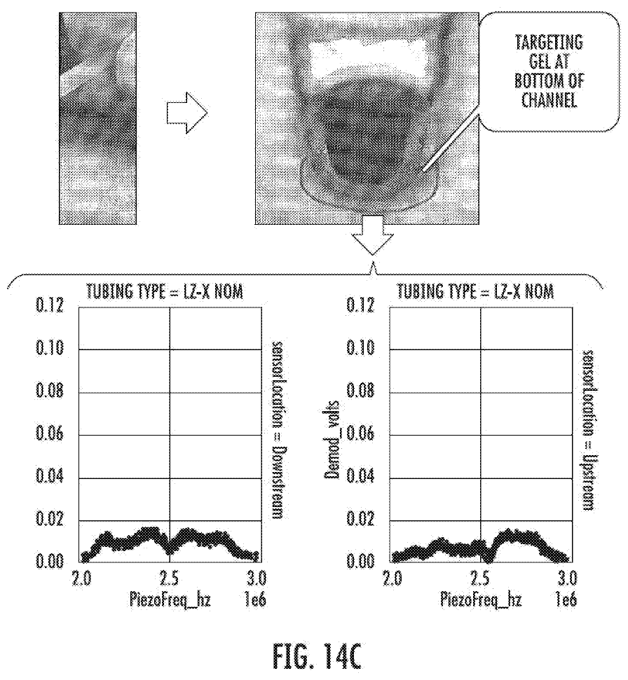

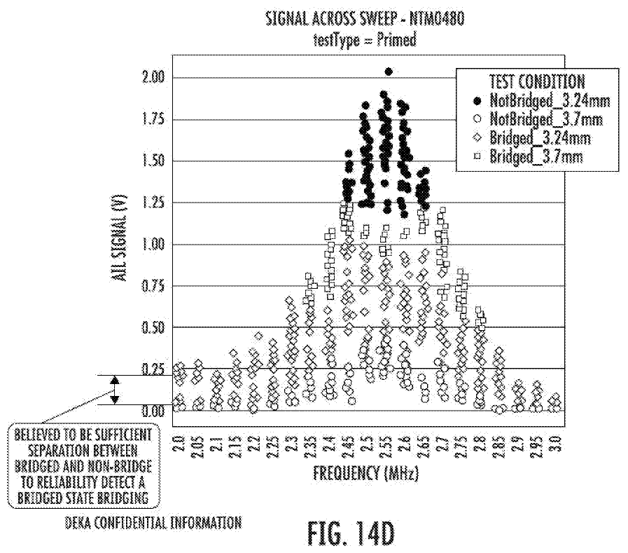

FIG. 14A illustrates a frequency-response plot for various sensor states. In some embodiments, the product operates at a single drive frequency selected as part of a per-sensor calibration process. The selected frequency may be stored in non-volatile memory and retrieved during system initialization. The memory may also store serial number data, tubing type, calibration date, frequency-response profile data, thresholds, gain values, bridge-detection thresholds, expected empty-response data, expected primed-response data, or other configuration parameters.

Communication between the controller, EEPROM, programmable oscillator, and programmable logic may be performed using an I2C bus, SPI bus, GPIO lines, memory-mapped registers, or other communication paths. The programmable logic may generate clocking and control signals for transmitter drive, sample windows, switching networks, integrator resets, sample-and-hold timing, and diagnostic routines.

In one non-limiting calibration procedure, the controller or an external calibration station drives the transmitter across a plurality of test frequencies while the conduit is in at least one known condition. The known condition may include an empty sensor, empty tubing, primed tubing, submerged tubing, a reference tube, a reference insert, or a simulated bridge. A response profile is generated and the calibrated drive frequency is selected based on a stored selection rule. The selection rule may prioritize separation between fluid and air at the operating frequency while maintaining detectability of bridge conditions at one or more diagnostic frequencies.

External bridging may occur when an acoustic coupling path exists outside the intended internal fluid path. For example, water, gel, residue, cleaning solution, condensation, or another material may bridge between portions of the sensor housing, between a transmitter and receiver, or around the conduit. Such a bridge may transmit ultrasonic energy even when the conduit itself is empty, thereby producing a false fluid-present indication if only an operational-frequency magnitude is considered.

FIGS. 14B and 14C illustrate example conditions in which gel or another coupling material is applied near different portions of a conduit channel. In one condition, a bridge at a top portion of the channel may produce a measurable frequency response, while a bridge at a bottom portion may produce a substantially different response. FIG. 14D illustrates example frequency-sweep data for bridged and non-bridged conditions. The illustrated data show that off-band responses may provide separation between bridged and non-bridged states.

In some embodiments, the controller transmits ultrasonic energy at a calibrated operational frequency and at one or more off-band frequencies. The off-band frequency may be outside a primary resonance band, below the operational band, above the operational band, or otherwise selected to distinguish normal through-fluid acoustic coupling from external bridging. Other frequency ranges may be used depending on sensor geometry, tubing, and transducer selection.

A bridging diagnostic may be performed during startup, before therapy, after door opening, after set loading, during priming, during maintenance, during a commanded self-test, periodically during therapy, after a cleaning prompt, or when a signal anomaly is detected. A known or expected empty-conduit condition may be established by an empty sensor state, an absent tubing state, an unprimed tubing state, a commanded purge state, a door-open state, a flow-stop state, another sensor input, or a user-commanded diagnostic state. If the off-band magnitude exceeds a threshold under the known or expected empty condition, the controller may infer a bridging condition.

In some embodiments, the controller performs a gated fluid-presence determination. A candidate fluid-present determination may first be generated when an operating-frequency magnitude exceeds a fluid threshold. The candidate determination is not asserted as a valid fluid-present state unless bridge logic also indicates that the measurement is reliable. The bridge logic may require that an off-band magnitude remain below a bridge threshold, that a ratio of off-band magnitude to operating-frequency magnitude remain below a ratio threshold, that a frequency-response profile not match a stored bridge profile, or that another sensor confirm the fluid-present condition.

In one example, the controller obtains an operating magnitude MOP at the calibrated drive frequency and obtains diagnostic magnitudes MD1 through MDN at N diagnostic frequencies. A bridge metric BM may equal max(MD1/T1, MD2/T2, . . . MDN/TN), where T1 through TN are corresponding diagnostic thresholds. If MOP exceeds a fluid threshold but BM also exceeds a bridge threshold, the controller may determine that the apparent fluid-present response is unreliable because an external acoustic bridge may be carrying signal energy along or around an exterior surface of the conduit instead of through the internal lumen. In another example, BM may equal an off-band-to-operating ratio, an off-band area under a response curve, a count of diagnostic frequencies exceeding thresholds, or a classifier score.

When a bridging condition is detected, the controller may generate an alarm, generate a bridge alarm, generate an air-detection-unreliable alarm, stop or inhibit pumping, instruct the user to clean or reload the set, record diagnostic data, or enter a maintenance mode. The controller may also prevent a fluid-present state from being used as an air-clear condition based solely on the calibrated-frequency magnitude. In some embodiments, the alarm is maintained until a bridge-clearing test, a reload operation, a cleaning confirmation, a diagnostic sequence, or another reliability check indicates that the ultrasonic path is again representative of the internal lumen.

The bridge-detection thresholds may be fixed, calibration-derived, tubing-type-derived, temperature-compensated, pressure-compensated, gain-compensated, or adaptive. For example, thresholds may be stored in non-volatile memory with the calibrated drive frequency. In another embodiment, thresholds may be updated based on a recent empty-tube baseline, a recent primed-tube baseline, or a diagnostic sweep conducted after a door-open or set-loading event.

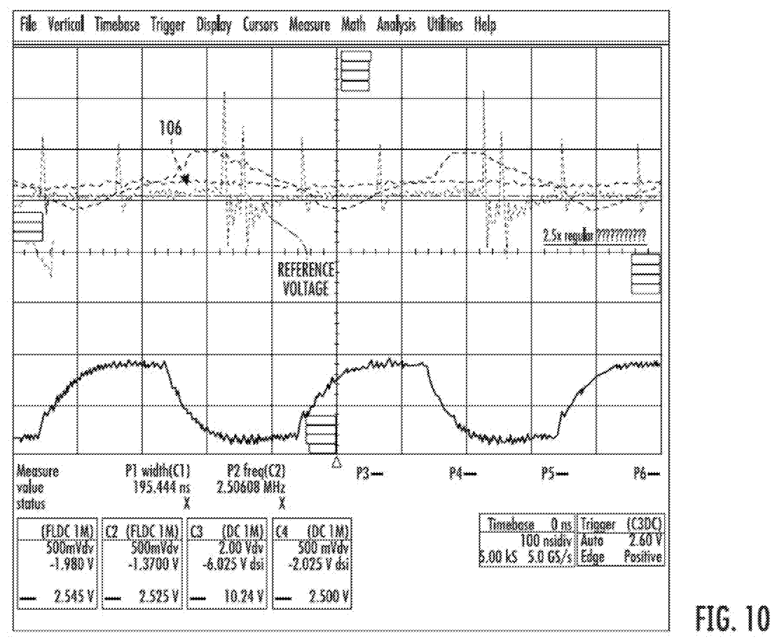

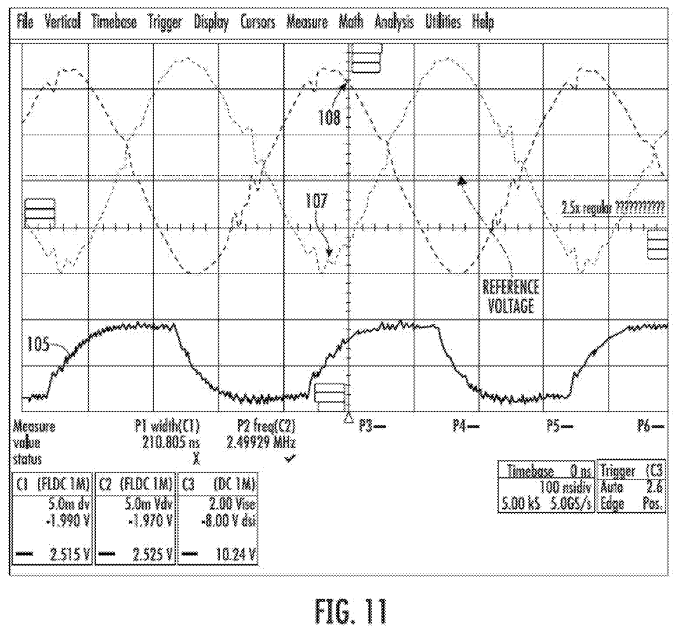

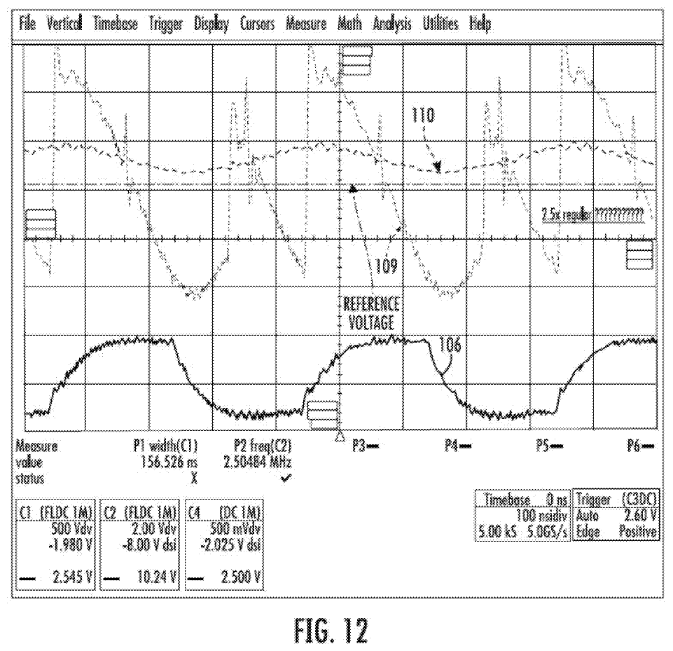

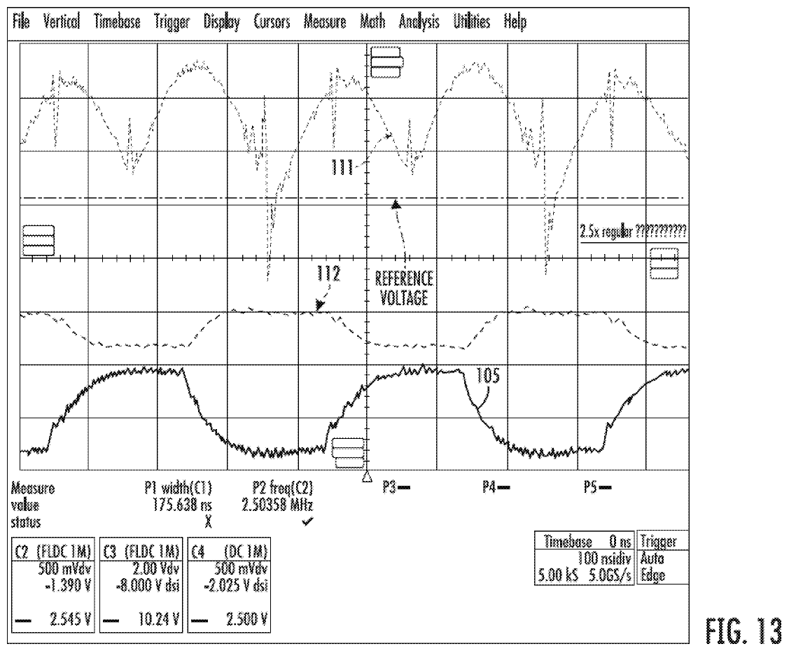

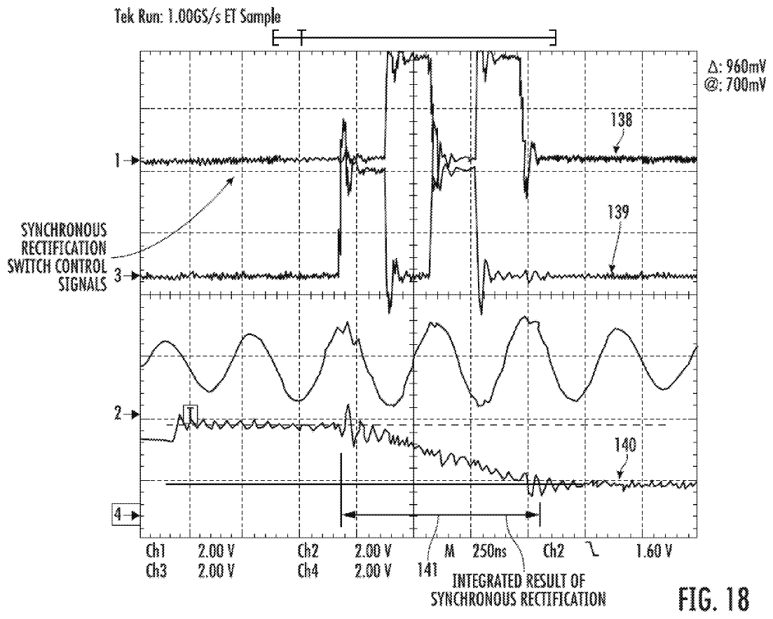

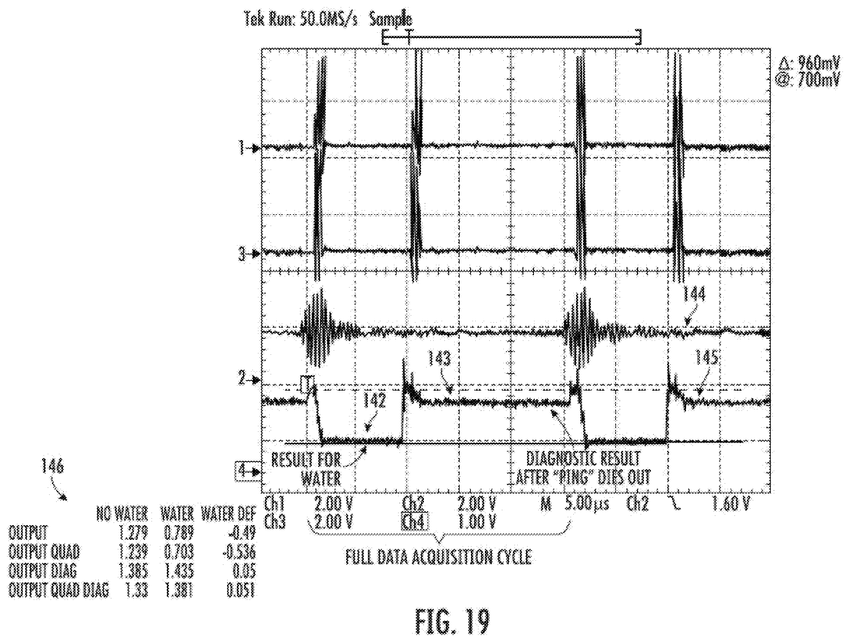

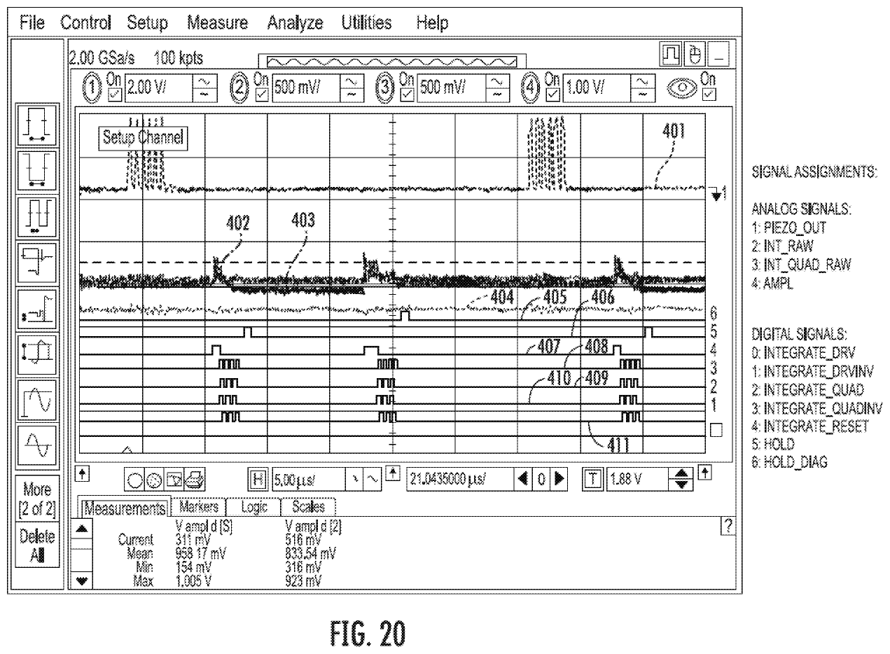

FIGS. 10-13 and 18-21 illustrate representative signal traces. A transmitter-drive signal may be applied in bursts or pings. A receiver signal may be processed by inverting and non-inverting amplifier paths. Synchronous rectification switch control signals may be timed relative to the drive signal, receiver signal, or a reference clock. Integrated outputs may be sampled during windows corresponding to the acoustic response and diagnostic baseline. Sample-and-hold outputs may be digitized by an ADC and processed by the controller.

A diagnostic portion of a signal may be sampled after a ping has substantially dissipated. Because the diagnostic portion may be dominated by offset rather than acoustic signal, the diagnostic sample may be used to correct or monitor amplifier offset, switch charge injection, comparator offset, integrator reset error, or other circuit artifacts. In some embodiments, when diagnostic values exceed high or low diagnostic thresholds, the controller determines that a fault condition exists and inhibits reliance on primary output values.

In some embodiments, the ultrasonic sensing system need not estimate continuous flow acoustically. Delivered volume and fluid movement may instead be determined from pump commands, motor position, cam position, plunger position, valve timing, pump cycle count, calibrated stroke volume, pressure-compensated volume, or another pump-control parameter. The ultrasonic sensing system may be used primarily to classify air, fluid, bridge, bubble, tube-loading, and sensor-reliability states, and those states may be used to validate pump operation, detect occlusions, detect free-flow conditions, estimate air volume, or determine whether an alarm condition should be generated.

Multiple acoustic paths may be used to improve spatial resolution, redundancy, bubble-size estimation, bubble-direction detection, or bridge detection. One arrangement includes one transmitter and multiple receivers. Another arrangement includes multiple transmitters and one receiver. Another arrangement includes multiple transmitters and multiple receivers. The transmitters may be aligned, anti-aligned, angled, staggered, or arranged to define upstream and downstream sensing paths. Crosstalk may be managed by time-division multiplexing, frequency-division multiplexing, code-division multiplexing, directional acoustic shielding, filtering, or combinations thereof.

The drawing numbers and reference characters used herein refer to the drawing set of the present application. Figures and reference characters used in any incorporated or related application may differ and do not control the numbering of the present drawings unless expressly stated.

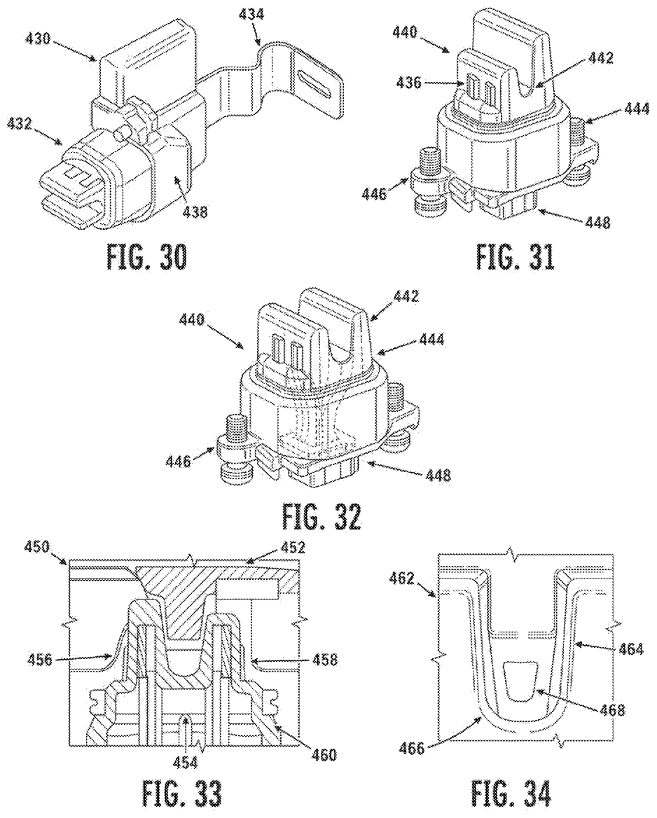

FIGS. 30-34 illustrate representative mechanical structures for positioning a fluid conduit relative to ultrasonic transducers. In FIG. 30, a sensor connector or sensor assembly 430 includes a connector body 432, a flexible lead, cable support, or mounting extension 434, and a latch or registration feature 438. The connector body 432 may couple electrical connections, a sensor housing, or a disposable-set feature to a pump, controller, cassette, or reusable sensing module. The flexible lead or mounting extension 434 may provide strain relief, keyed mounting, alignment, or electrical routing, and the latch or registration feature 438 may hold the assembly 430 in a repeatable position.

In FIGS. 31 and 32, a sensor clamp assembly 440 includes a mounting or alignment tab 436, a tube-receiving saddle or channel 442, a transducer support structure 444, one or more fasteners, springs, or biasing members 446, and a base or cassette-interface structure 448. The tube-receiving saddle or channel 442 may locate a tube relative to the ultrasonic transducer arrangement. The transducer support structure 444 may support an ultrasonic transmitter, receiver, transceiver, acoustic window, potting compound, matching layer, or acoustic isolation feature. The fastener, spring, or biasing member 446 may establish clamping force, preload, or positional repeatability. The base or cassette-interface structure 448 may register the sensor clamp assembly 440 with a pump housing, cassette, disposable set, door, platen, or flow-stop component.

In FIG. 33, a fluid conduit 450 is positioned between an upper clamp member 452 and a lower conduit support 454. A first ultrasonic element or support 456 and a second ultrasonic element or support 458 are positioned on opposite sides of the conduit 450. A controlled compression or contact region 460 is established so that the conduit 450 is repeatably shaped and acoustically coupled without excessive occlusion. The contact region 460 may be used to set acoustic path length, contact pressure, conduit geometry, or acoustic coupling, and may be coordinated with calibration data or pressure compensation.

In FIG. 34, a conduit-receiving channel or groove 462 includes a sidewall 464, a bottom support 466, and a relief, drainage, vent, hydrophobic, or cleaning feature 468. The sidewall 464 and bottom support 466 may constrain conduit position. The relief, drainage, vent, hydrophobic, or cleaning feature 468 may reduce retention of bridge-forming material such as water, gel, residue, medication, biological fluid, condensate, or cleaning solution. The feature 468 may therefore cooperate with the bridge-diagnostic routines described herein to reduce false fluid-present determinations caused by external acoustic bridging.

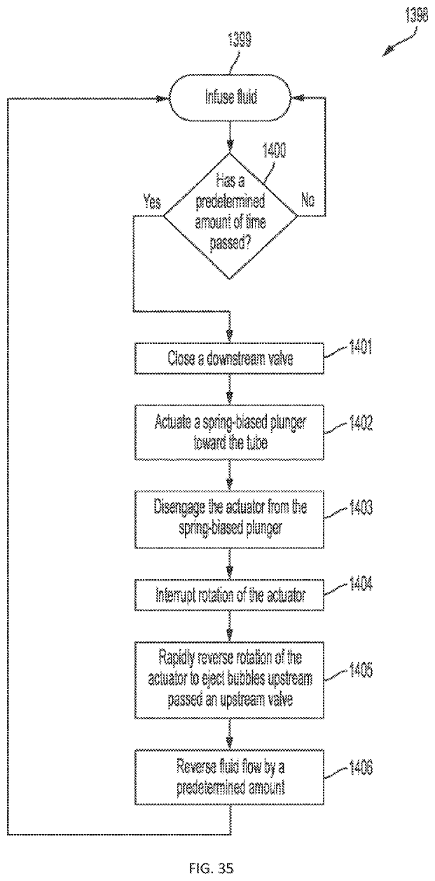

FIG. 35 illustrates a bubble-displacement method 1398. During an infusion operation 1399, a controller determines at decision 1400 whether a predetermined amount of time, delivered volume, pump cycles, or other interval has passed. If so, the controller closes a downstream valve at operation 1401, actuates a spring-biased plunger toward the tube at operation 1402, disengages the actuator from the spring-biased plunger at operation 1403, interrupts rotation or motion of the actuator at operation 1404, rapidly reverses actuator rotation or motion to eject bubbles upstream at operation 1405, and reverses fluid flow by a predetermined amount at operation 1406.

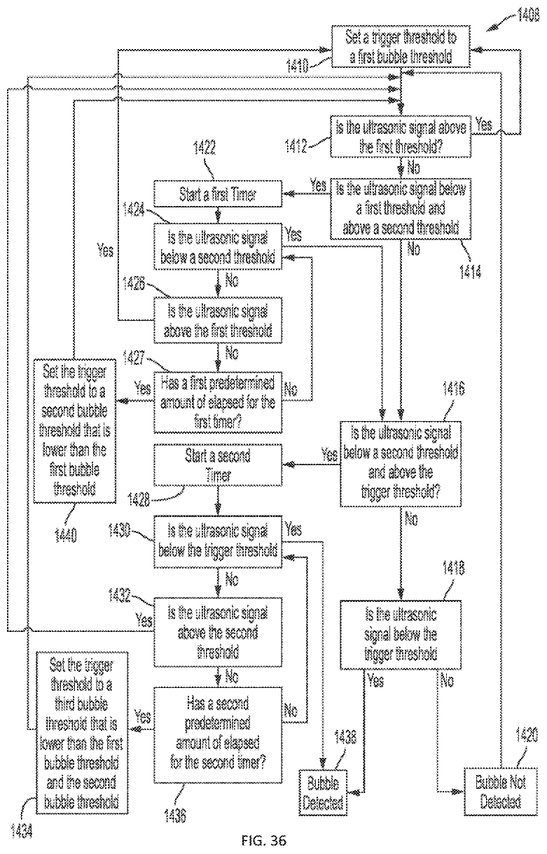

FIG. 36 illustrates a multi-threshold bubble-detection method 1408. The method may set a trigger threshold to a first bubble threshold at operation 1410, compare an ultrasonic signal to a first threshold at decision 1412, compare the signal to an intermediate threshold range at decision 1414, and compare the signal to a second threshold or trigger threshold at decisions 1416 and 1418. A bubble-not-detected state 1420 may be entered when criteria are not satisfied. A first timer may be started at operation 1422, second-threshold and first-threshold comparisons may be performed at decisions 1424 and 1426, and a first elapsed-time decision 1427 may determine whether to lower the trigger threshold. A second timer may be started at operation 1428, a trigger-threshold comparison may be performed at decision 1430, a second-threshold comparison may be performed at decision 1432, a lower third bubble threshold may be set at operation 1434, a second elapsed-time decision may be performed at decision 1436, a bubble-detected state 1438 may be asserted, and the trigger threshold may be set to a second bubble threshold at operation 1440.

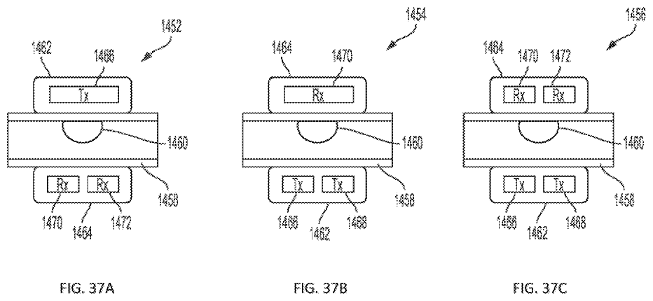

FIGS. 37A-37C illustrate multi-path ultrasonic sensor arrangements. Arrangement 1452 may include a transmitter Tx and first and second receivers Rx. Arrangement 1454 may include first and second transmitters Tx and a receiver Rx. Arrangement 1456 may include multiple transmitters and multiple receivers. A tube or fluid conduit region 1458 and a fluid, air, or bubble region 1460 are positioned relative to transducer supports 1464. Transmitters 1462 and 1468 and receivers 1470 and 1472 may define upstream and downstream acoustic paths, aligned paths, or redundant paths. A controller may use the multiple paths to estimate bubble position, velocity, transit time, direction, volume, path agreement, or sensor reliability.

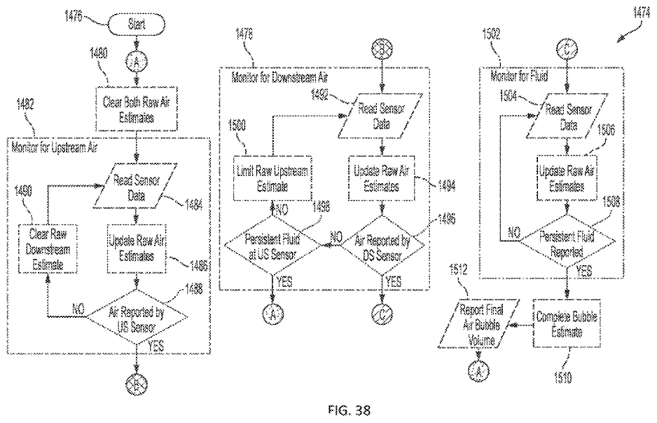

FIGS. 38-40 illustrate multi-path bubble-estimation routines. In FIG. 38, a bubble-estimation routine 1474 begins at start state 1476. The controller may clear raw air estimates at operation 1480, monitor an upstream air path at block 1482, read upstream sensor data at operation 1484, update raw upstream air estimates at operation 1486, determine whether air is reported by the upstream sensor at decision 1488, and clear a raw downstream estimate at operation 1490. The controller may monitor downstream air at block 1478, read downstream sensor data at operation 1492, update raw downstream air estimates at operation 1494, determine whether air is reported by the downstream sensor at decision 1496, determine persistent fluid at decision 1498, limit a raw upstream estimate at operation 1500, monitor for fluid at block 1502, read sensor data at operation 1504, update raw air estimates at operation 1506, determine persistent fluid at decision 1508, complete a bubble estimate at operation 1510, and report a final air-bubble volume at operation 1512.

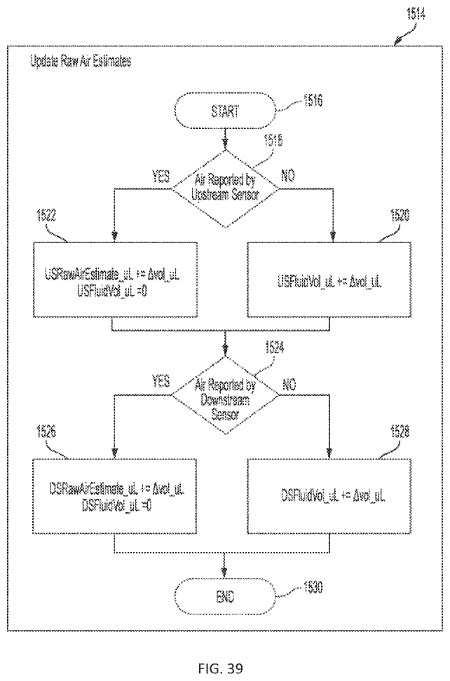

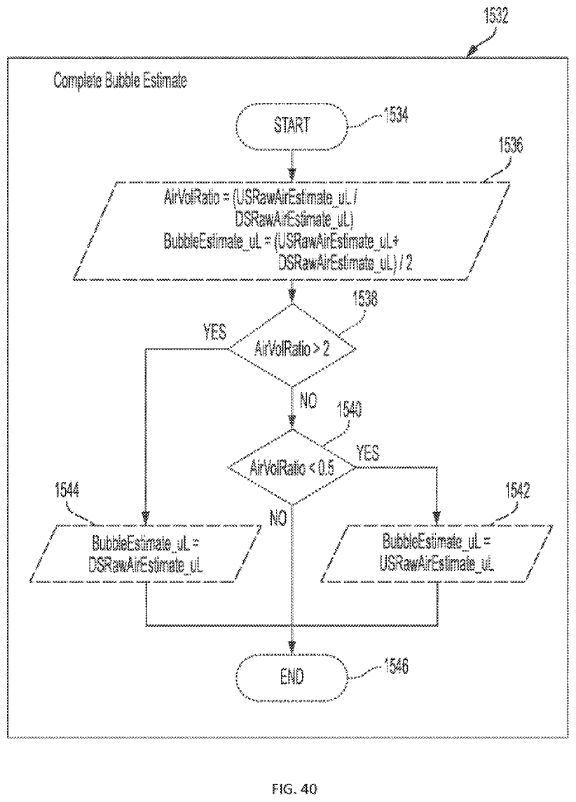

In FIG. 39, an update routine 1514 begins at start state 1516, determines whether air is reported by the upstream sensor at decision 1518, updates an upstream fluid volume at operation 1520 when fluid is reported, updates an upstream raw air estimate at operation 1522 when air is reported, determines whether air is reported by the downstream sensor at decision 1524, updates a downstream raw air estimate at operation 1526 when air is reported, updates a downstream fluid volume at operation 1528 when fluid is reported, and terminates at end state 1530. In FIG. 40, a complete-bubble-estimate routine 1532 begins at start state 1534, calculates an air-volume ratio at operation 1536, compares the ratio with an upper bound at decision 1538 and a lower bound at decision 1540, selects an upstream raw air estimate at operation 1542 or a downstream raw air estimate at operation 1544 when appropriate, and terminates at end state 1546.

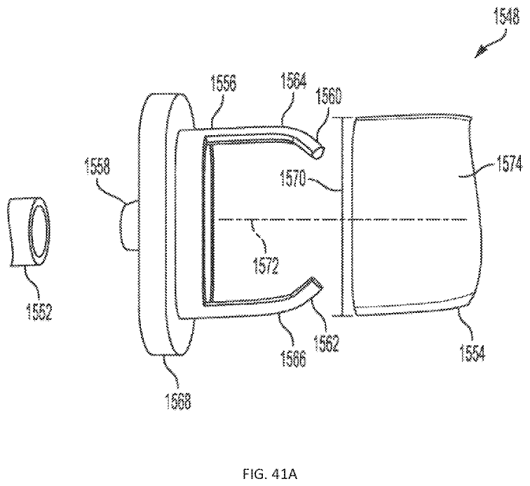

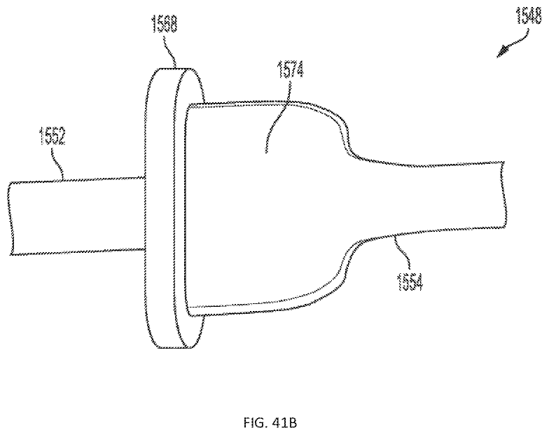

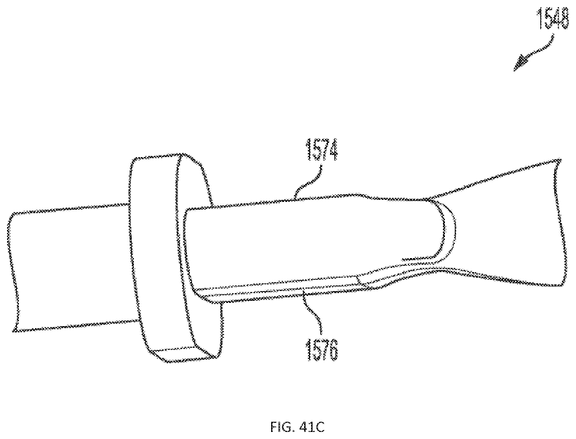

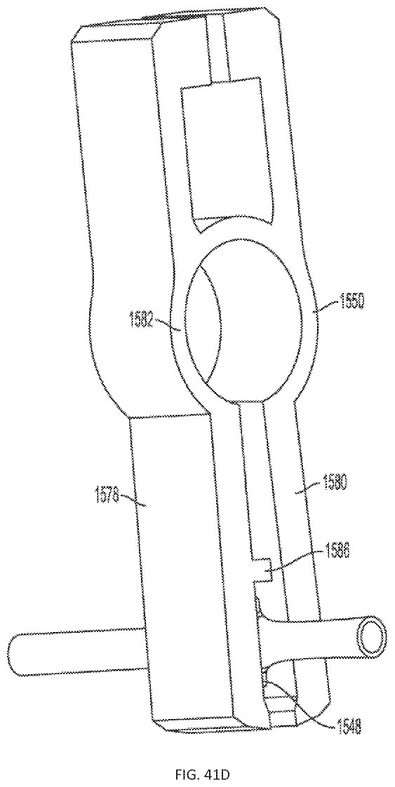

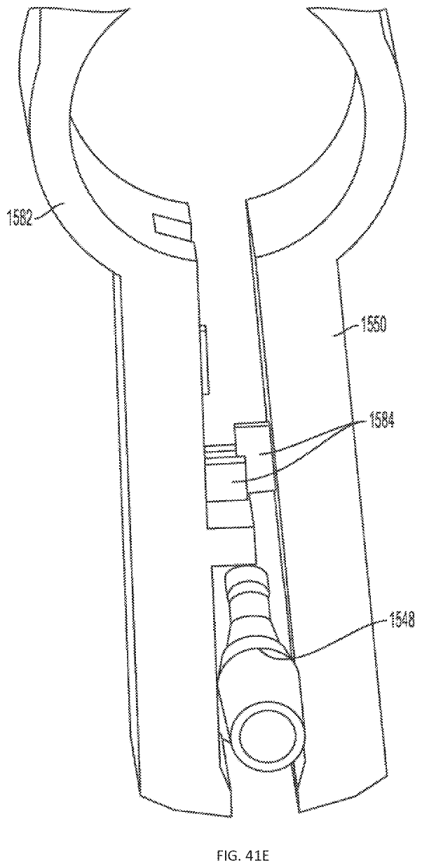

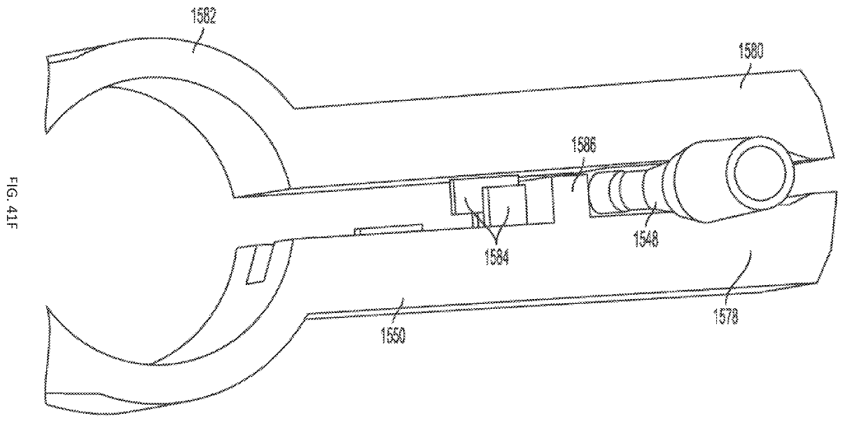

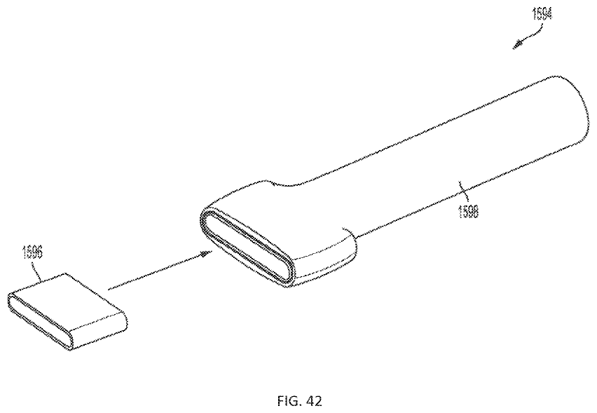

FIGS. 41A-41F and FIG. 42 illustrate pressure-sensing, pressure-management, and conduit-deformation structures. An in-line pressure sensor, clip, or sensor assembly 1548 may include a sensor opening, port, insert, or pressure interface 1550, an upstream or first tube section 1552, a downstream or second tube section 1554, clip arms or elongated supports 1556 and 1566, a sensor, insert, or clip component 1558, a tube-receiving opening 1560, a plunger, actuator, or pressure interface 1562, an elongated member 1564, a flange or clip body 1568, a clamp insert or interface wall 1570, a longitudinal axis 1572, a downstream bladder or tube section 1574, a compliant wall or bladder region 1576, first and second clip portions 1578 and 1580, a groove or deformation feature 1582, coupling or engagement features 1584 and 1586, a sensor support or interface feature 1588, and a narrowed or pressure-sensitive tube region 1590. In FIG. 42, an insert 1596 may be positioned in a fluid-bearing tube section 1598 of a pressure-sensing assembly 1594.

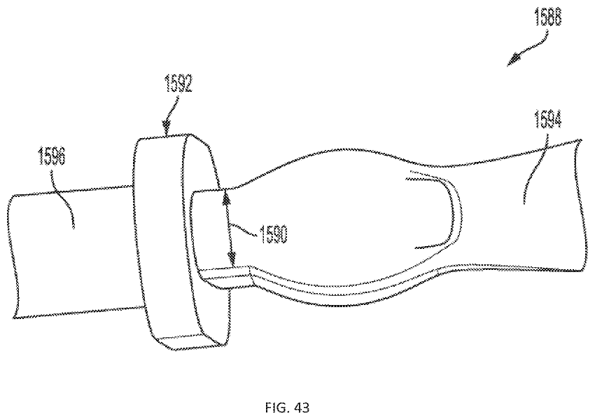

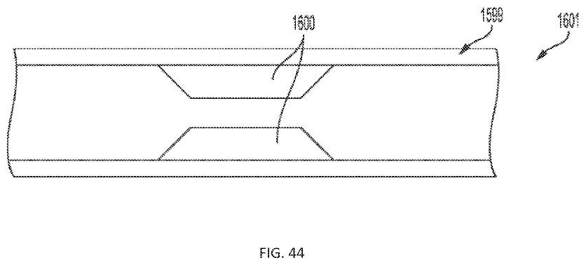

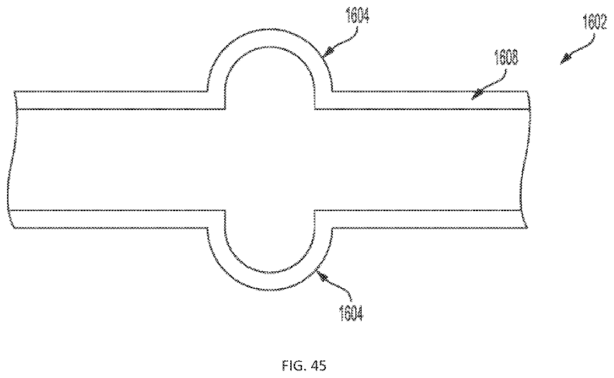

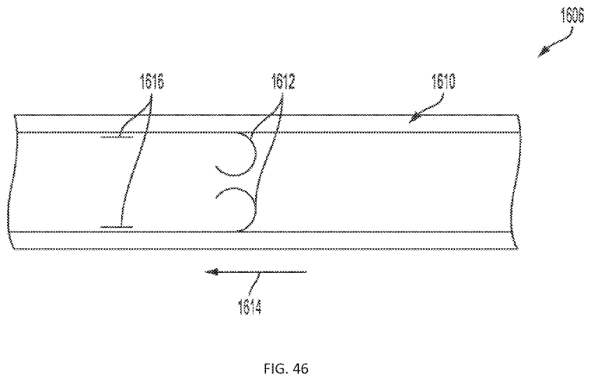

FIGS. 43-46 illustrate pressure-management and bubble-management features. A compliant bladder or pressure-management assembly 1588 may include a coupler or flange 1592, a tube or outlet section 1594, an upstream or inlet tube section 1596, and a narrowed or pressure-sensitive region 1590. A tube section 1601 may include an impeder, narrowed region, or restriction 1600. A compliant tube or bladder assembly 1602 may include one or more compliant wall or bladder regions 1604 and a fluid conduit 1608. A tubing section 1606 may include a tube section 1610, nucleation sites 1612, a flow direction 1614, and an air trap or collection feature 1616.

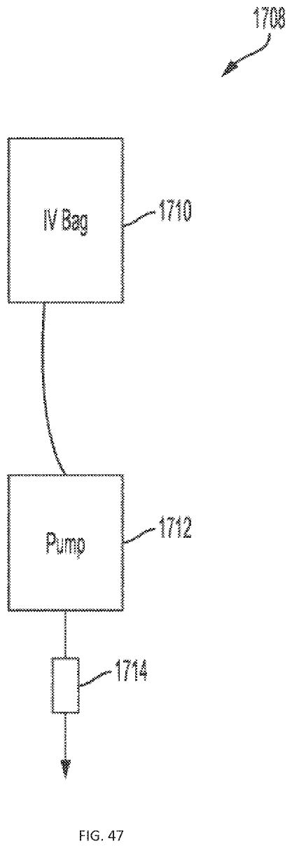

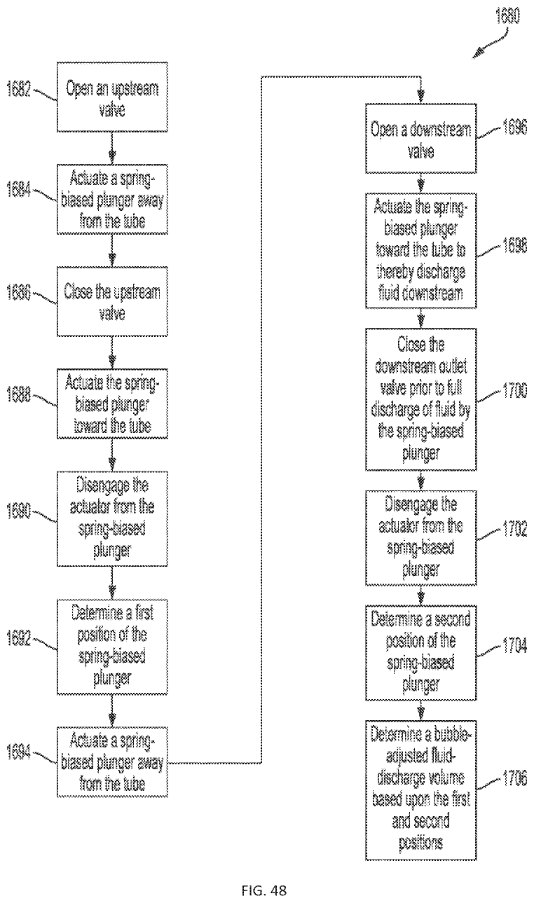

FIG. 47 illustrates a pressure-maintaining fluid-delivery system 1708 including an IV bag 1710, a pump 1712, and a check valve 1714. The check valve 1714 may maintain a downstream pressure above an outgassing threshold, reduce bubble formation, or improve delivered-volume and air-volume estimates. FIG. 48 illustrates a delivered-volume compensation routine 1680 in which an upstream valve is opened at operation 1682, a spring-biased plunger is actuated away from a tube at operation 1684, the upstream valve is closed at operation 1686, the spring-biased plunger is actuated toward the tube at operation 1688, an actuator is disengaged at operation 1690, a first plunger position is determined at operation 1692, the spring-biased plunger is actuated away from the tube at operation 1694, a downstream valve is opened at operation 1696, the spring-biased plunger is actuated toward the tube to discharge fluid downstream at operation 1698, a downstream outlet valve is closed before full discharge at operation 1700, the actuator is disengaged at operation 1702, a second plunger position is determined at operation 1704, and a bubble-adjusted fluid-discharge volume is determined at operation 1706 based on the first and second positions.

The pressure, outgassing, bubble-displacement, and delivered-volume-compensation embodiments described with respect to FIGS. 35-48 may be used independently or in combination with the calibrated operating-frequency and off-band bridge-detection embodiments. For example, a bridge-detection routine may determine whether an air detector is reliable, a multi-threshold bubble-detection routine may determine whether an air event occurred, a multi-path estimation routine may estimate air volume, and a delivered-volume compensation routine may adjust a therapy-volume estimate. The same controller, or distributed controllers communicating over a pump bus, may perform these operations.

Although particular circuits are described, the functions may be implemented using analog circuitry, digital circuitry, mixed-signal circuitry, a microcontroller, a digital signal processor, an FPGA, a CPLD, an ASIC, discrete components, software, firmware, or combinations thereof. A controller may compute a magnitude directly from digitized I and Q samples, from integrated outputs, from filtered outputs, from comparator states, from lookup tables, or from a trained classifier. Statistical models may be used to classify air, fluid, bridge, particulate, foam, or artifact states where sufficient training data and validation are available.

The terms transmitter and receiver are used for clarity. In some embodiments, the same piezoelectric element may act as both transmitter and receiver at different times. A sensor may include more than two transducers. A pump may include a peristaltic pump, syringe pump, ambulatory pump, modular pump, enteral pump, dialysis pump, or another fluid delivery device.

In some embodiments, the ultrasonic sensing functions described herein may be implemented as a stand-alone air-in-line sensor, as a sensor integrated into a pump housing, as a sensor integrated into a pump door or platen, as a sensor integrated into a disposable administration set or cassette, as a sensor integrated into a flow-stop component, or as a sensor coupled to a gravity-fed infusion line. The same detection architecture may be used with large-volume pumps, ambulatory pumps, syringe pumps, insulin pumps, dialysis devices, enteral feeding systems, parenteral nutrition systems, transfusion systems, drug-specific delivery systems, and modular medical pump systems.

In some embodiments, the signal-generation circuit may drive the ultrasonic transmitter using bursts, pings, continuous-wave signals, swept-frequency signals, chirps, coded signals, phase-coherent signals, time-multiplexed signals, frequency-multiplexed signals, or code-multiplexed signals. The transmitted signal may be generated by a crystal oscillator, programmable oscillator, direct digital synthesizer, numerically controlled oscillator, phase-locked loop, microcontroller timer, FPGA, CPLD, ASIC, discrete timing circuit, or another clock-generation circuit. The same or different drive circuitry may be used for normal air/fluid discrimination, bridge diagnostics, alarm-condition detection, and self-test.

In some embodiments, the received signal may be processed using analog demodulation, digital demodulation, synchronous rectification, active rectification, envelope detection, amplitude detection, phase detection, frequency-response analysis, decay-time analysis, ringing analysis, quality-factor analysis, harmonic analysis, correlation processing, matched filtering, or machine-classification processing. A controller may classify sensor states including fluid, air, empty tube, absent tube, unprimed tube, primed tube, foam, particulate, bridge, contamination, sensor fault, tube misload, occlusion, free-flow, and unknown or low-confidence states.

In some embodiments, a calibration routine may select one or more operating frequencies and one or more diagnostic frequencies based on a response profile measured under known conditions. The known conditions may include an empty sensor condition, an absent-tubing condition, an empty-tubing condition, a primed-tubing condition, a submerged condition, a partially filled condition, a simulated bridge condition, a known contaminant condition, a pressure-loaded condition, a temperature-loaded condition, or a reference insert condition. Stored calibration data may include a serial number, a set identifier, tube size, tube material, tube compliance, tube lot, operating frequency, diagnostic frequency, gain, phase offset, baseline, thresholds, response profile, expected empty response, expected primed response, expected bridged response, temperature coefficients, pressure coefficients, and service history.

In some embodiments, external acoustic bridging may be detected using a bridge metric based on one measurement or a combination of measurements. The bridge metric may use an off-band magnitude, an off-band-to-operating-frequency ratio, an off-band difference from an expected empty response, a frequency-response area, a frequency-response slope, a peak width, a peak shift, a peak count, a decay time, a ringing amplitude, a phase shift, a correlation score, a classifier score, an agreement score across multiple acoustic paths, or a count of diagnostic frequencies satisfying corresponding thresholds. Bridge logic may be performed during startup, before therapy, after set loading, after a door-open event, after a latch event, during priming, during purge, after a cleaning prompt, periodically during therapy, after a signal anomaly, and before re-enabling therapy after a fault.

In some embodiments, bridge-forming material may include water, gel, coupling material, cleaning solution, detergent, residue, medication, biological fluid, blood, condensate, humidity, adhesive, lubricious coating, plasticizer, tube residue, particulate matter, material trapped between a tube and a housing, material wicked into a channel, or material spanning a transmitter-receiver gap outside the internal lumen. The bridge may occur around the conduit, along a housing wall, across a clamp feature, through a potting compound, through a disposable set component, through a door or platen surface, through a flow-stop interface, or along another acoustic path that bypasses the intended fluid-containing lumen.

In some embodiments, a valid fluid-present state is produced only after a candidate fluid-present state has been qualified by one or more reliability checks. The reliability checks may include bridge rejection, diagnostic-window validation, comparator-window validation, saturation detection, offset detection, gain-range validation, tube-position validation, pressure validation, temperature validation, agreement among acoustic paths, agreement with optical, pressure, capacitance, conductivity, motor-position, valve-state, door-state, flow-stop, or set-identification sensors, and agreement with an expected pump state.

In some embodiments, the mechanical support for the fluid conduit may include a V-groove, U-groove, arcuate groove, tapered channel, tube nest, tube guide, resilient pad, spring-biased clamp, hard stop, flow-stop interface, cassette interface, platen interface, registration pin, latch, door feature, alignment rib, drainage channel, vent, wicking-break, hydrophobic surface, oleophobic surface, textured surface, low-surface-energy coating, acoustic isolation gap, relief channel, or seal. These features may position the conduit repeatably, reduce tolerance stack-up, avoid excessive occlusion, reduce retained bridge-forming material, and preserve a reliable acoustic path through the intended sensing region.

In some embodiments, the transmitter and receiver may be separate piezoelectric elements, a common piezoelectric element used at different times, multiple transmitters, multiple receivers, opposed pairs, angled pairs, staggered pairs, aligned pairs, anti-aligned pairs, upstream and downstream pairs, longitudinal pairs, transverse pairs, or combinations thereof. The transducers may be mounted in a reusable housing, mounted in a disposable component, encapsulated in epoxy, overmolded in polymer, retained by adhesive, retained by potting compound, supported by elastomer, mounted to a printed circuit board, mounted to a flex circuit, or acoustically coupled through a window or matching layer.

In some embodiments, air may be tracked as a therapy-session total, a rolling-window total, a predetermined-interval total, a single-event amount, an event count, an event duration, an estimated volume, a compensated volume, a selected upstream/downstream estimate, a confidence-weighted estimate, or a cumulative value associated with a drug-delivery limit. The controller may compare the air metric with one or more thresholds and may generate alarms or pump actions based on a single bubble, a cluster of bubbles, persistent air, foam, repeated marginal events, rate of air accumulation, or an amount of air within a most-recent interval.

In some embodiments, a bubble event may be defined using a fluid-clear volume, a hold-up volume, an upstream air-reporting condition, a downstream air-reporting condition, and one or more delivered-delta volumes. Delivered delta volumes may be based on pump motor position, cam position, plunger position, valve timing, pump cycle count, commanded stroke volume, calibrated stroke volume, measured flow, pressure-compensated volume, calibration tables, or another volume source. Upstream and downstream raw air estimates may be averaged, selected, weighted, capped, discarded, or faulted based on a ratio, confidence measure, transit time, or agreement rule.

In some embodiments, multi-threshold bubble logic may include first and second bubble thresholds, an adjustable trigger threshold, a reset threshold, a first timer, a second timer, debounce logic, hysteresis, persistence criteria, and noise rejection logic. The trigger threshold may be lowered after a signal remains within an intermediate range for a predetermined time, raised after persistent fluid is detected, reset after a new pump cycle, reset after a bridge diagnostic indicates an unreliable measurement, or adjusted based on tube type, temperature, pressure, flow rate, fluid type, and calibration data.

In some embodiments, a pump may respond to an air or bridge condition by stopping infusion, closing a valve, inhibiting pumping, pausing therapy, reversing an actuator, reversing flow, dislodging a bubble, increasing line pressure, maintaining a valve state, modifying a delivered-volume estimate, derating a confidence value, requiring re-priming, requiring set reloading, prompting cleaning, prompting maintenance, logging diagnostic data, communicating with a pump user interface, communicating with a service tool, communicating with a hospital information system, or communicating with a remote monitoring system.

In some embodiments, a bubble-displacement routine may close a downstream valve, interrupt or disengage a spring-biased plunger or another pump actuator, reverse rotation or motion of the actuator, move fluid upstream, and eject bubbles past an upstream valve. The routine may be performed after a predetermined time, a predetermined volume, a predetermined number of pump cycles, a detected downstream-air amount, a detected upstream-air amount, a priming operation, a maintenance operation, or a pre-therapy check.

In some embodiments, pressure-related structures may be used with the air detector. A pressure sensor may include a clip, elongated members, pivoting members, a flattened tube region, an insert, a port, a tube deformation region, an optical displacement sensor, magnetic displacement sensor, strain sensor, force sensor, capacitive sensor, inductive sensor, Hall sensor, or another pressure-responsive sensor. Pressure may be used to compensate ultrasonic thresholds, detect occlusion, detect free-flow, estimate outgassing likelihood, refine bubble-volume estimates, refine delivered-volume estimates, select bridge thresholds, or control pressure-management features.

In some embodiments, pressure-management and air-management features may include a compliant tube section, a bladder, an accumulator, an impeder, a restriction, a reduced-diameter region, a textured region, a nucleation site, an air trap, a gas collection chamber, a vent, a hydrophobic membrane, a heater, a check valve, or a pressure-maintaining valve. A check valve may provide a fixed or adjustable crack pressure selected to maintain a line-pressure nadir above an outgassing pressure. Nucleation sites may be molded, etched, roughened, scored, textured, laser-formed, chemically treated, or otherwise formed to promote controlled bubble formation at a selected location upstream of an air trap or air detector.

In some embodiments, the ultrasonic sensing system may cooperate with a pump platform having a door, a lever, a cam, a shaft, a carriage, a tube-receiving region, a platen, a flow stop, a flow-stop carriage, a latch, a catch, a spring, a motor, a gear train, a pump actuator, a plunger, a valve, a flow-stop identification feature, a sensor board, an optical emitter, an optical receiver, a light pipe, and a controller. The controller may use door state, lever state, latch state, flow-stop identification, set identification, motor state, valve state, pump cycle state, and tube-loading state to determine when to perform an air-detection routine, bridge-detection routine, priming routine, pressure-management routine, or delivered-volume compensation routine.

In some embodiments, a disposable set or flow-stop component may include a tube segment, a flange, a shutter, a slot, an aperture, a light-transmissive feature, an identifier hole pattern, a keyed geometry, a tube coupling, a flattened pressure-sensing region, a compliant chamber, an impeder, a nucleation site, an air trap, and a surface configured to register with a pump carriage. A pump may optically read the identifier hole pattern or another set-identification feature and select ultrasonic operating parameters, bridge thresholds, flow-calibration data, pressure-calibration data, and air-volume thresholds based on the identified set.

In some embodiments, any of the ultrasonic air-detection, bridge-detection, flow-estimation, pressure-sensing, pump-control, set-identification, disposable-set, and pump-platform features described herein may be used independently or in any technically compatible combination. For example, a pump may use set identification to select a calibration profile, use a door-open condition to initiate an empty-conduit bridge test, use quadrature demodulation to classify fluid presence, use diagnostic sample windows to reject offset, use multiple acoustic paths to estimate air volume, use pressure sensing to adjust thresholds, use a check valve to reduce outgassing, and use delivered-volume compensation to update a therapy-volume estimate.

In some embodiments, the controller may be implemented as one controller, multiple distributed controllers, a pump controller, a sensor controller, a disposable-set memory, a service tool, a calibration station, or a remote computing system. Software or firmware instructions may cause the controller to perform the functions described herein. The instructions may be stored in non-transitory memory, including flash memory, EEPROM, FRAM, RAM, ROM, memory within a microcontroller, removable memory, or memory associated with a server or service tool.

In some embodiments, the controller maintains a sensor-reliability state separate from an air/fluid state. The sensor-reliability state may include a reliable state, a bridge-detected state, a diagnostically invalid state, an unknown state, a cleaning-required state, a reload-required state, a service-required state, or a combination thereof. A fluid-present state may be output to, or used by, a therapy-control routine only when the sensor-reliability state indicates that the ultrasonic path is reliable.

In some embodiments, the controller performs the bridge-diagnostic sequence during, or immediately after detecting, a known or expected empty-conduit condition. The known or expected empty-conduit condition may be established by a door-open signal, a door-close signal following set loading, a latch signal, a flow-stop signal, an optical set sensor, a pressure sensor, a motor state, a priming command, a purge command, an absent-tube indication, an unprimed-tube indication, a service command, or another parameter indicating that liquid should not acoustically couple across the intended internal lumen.

In some embodiments, a diagnostic frequency is selected to expose acoustic coupling by material outside the internal lumen rather than merely to determine an optimum normal bubble-detection frequency. A diagnostic response may be treated as bridge-indicative when a diagnostic magnitude, ratio, phase, slope, decay time, peak width, peak position, path-agreement value, or other response feature differs from an expected empty response by more than a corresponding threshold.

In some embodiments, a calibration routine selects or validates an operating frequency and at least one diagnostic frequency by comparing response profiles acquired under two or more known conditions selected from an empty sensor condition, an absent-tube condition, an empty-tubing condition, a primed-tubing condition, a submerged-tubing condition, a simulated bridge condition, a known contaminant condition, a pressure-loaded tubing condition, a temperature-loaded tubing condition, and a reference-insert condition. The diagnostic frequency may be selected by maximizing separation between an expected empty response and an externally bridged response while maintaining a desired relationship to a primed response.

In some embodiments, the operating frequency may be within a range of about 2.0 MHz to about 3.0 MHz, or another ultrasonic range suitable for the selected transducers and tubing. A diagnostic frequency may be above, below, or otherwise spaced from the operating frequency by at least about 10 kHz, 25 kHz, 50 kHz, 100 kHz, 1%, 2%, 5%, 10%, or another offset selected during calibration. The foregoing numerical ranges are examples, and other frequencies and offsets may be used depending on transducer, conduit, housing, and fluid characteristics.

In some embodiments, set-identification information is used to select bridge-diagnostic parameters. The set-identification information may be obtained from an optical code, aperture pattern, RFID device, memory device, shape code, keyed geometry, barcode, user input, service tool, or pump configuration. The selected parameters may include an operating frequency, one or more diagnostic frequencies, bridge thresholds, empty-response profiles, primed-response profiles, gain settings, pressure compensation, temperature compensation, and air-volume thresholds.

In some embodiments, bridge detection is coordinated with physical anti-bridging features. Such features may include air gaps, acoustic isolation gaps, drainage gutters, weep channels, vent openings, hydrophobic coatings, oleophobic coatings, low-surface-energy coatings, texture transitions, relief channels, seals, barriers, or wicking breaks positioned to reduce retained bridge-forming material near a transmitter-receiver path. A diagnostic sequence may verify whether residual material remains after such features drain or redirect liquid.

In some embodiments, when a bridge criterion is satisfied, the controller can continue to track a candidate fluid-present state internally while outputting an alarm, invalid, unreliable, bridge-fault, cleaning-required, reload-required, or service-required state. The controller may re-enable assertion of a valid air-clear or fluid-present state only after a subsequent bridge-diagnostic sequence fails to satisfy the bridge criterion, after a cleaning or reloading procedure, or after confirmation from another sensor or modality.

In some embodiments, the bridge criterion is evaluated in combination with operating-frequency response data. For example, the controller may identify a bridge when an operating-frequency magnitude would otherwise indicate fluid while diagnostic response data obtained at one or more off-band frequencies corresponds to an externally bridged empty-conduit profile. In other embodiments, a bridge may be identified solely from diagnostic response data during an expected empty-conduit condition.

In some embodiments, the bridge-diagnostic operation is distinct from a normal bubble-detection or air/fluid discrimination operation. A normal bubble-detection operation may use an operating frequency and a fluid-present criterion selected to discriminate liquid in the lumen from gas in the lumen, while the bridge-diagnostic operation may use one or more diagnostic frequencies, diagnostic windows, bridge criteria, or expected-state checks selected to expose acoustic coupling outside the lumen. Thus, a signal that would otherwise satisfy the fluid-present criterion at the operating frequency may be withheld as an air-clear indication when the diagnostic response satisfies a bridge criterion or otherwise is inconsistent with a reliable through-lumen acoustic path.

In some embodiments, the controller maintains a bridge state machine. The bridge state machine may include an untested state, a bridge-suspected state, a bridge-detected state, a bridge-cleared state, and a sensor-disabled state. Transitions among the states may depend on expected empty-conduit tests, repeated off-band measurements, user cleaning confirmation, reloading of a disposable set, door/latch cycling, pressure or temperature stabilization, agreement with a second sensing modality, or successful completion of a bridge-clearing test.

In some embodiments, a bridge-clearing test includes repeating the bridge-diagnostic operation after a user cleaning prompt, after a set reload, after a purge sequence, after a dwell period, after a drying period, or after a pump door has been opened and closed. Therapy may remain inhibited, or an alarm may remain active, until the bridge metric falls below a clearing threshold. The clearing threshold may be the same as, lower than, or higher than the bridge-detection threshold to provide hysteresis.

In some embodiments, the controller distinguishes an external bridge from actual liquid in the conduit based at least in part on a contradiction between the ultrasonic response and an independent expected state. For example, a bridge may be inferred when an ultrasonic response indicates coupling while a door-open sensor, set-loading sensor, flow-stop sensor, optical sensor, pressure sensor, motor-position state, valve state, priming state, or user-commanded self-test indicates that a reliable fluid-present state should not yet be asserted.

In some embodiments, bridge criteria are established during manufacturing by measuring at least one unbridged empty-conduit response and at least one bridged or simulated-bridge response. The bridged or simulated-bridge response may be produced using water, gel, cleaning solution, saline, detergent, residue, a film, a wetted test fixture, a coupling pad, a reference insert, or another known acoustic-coupling condition. A calibration station may select operating and diagnostic frequencies, thresholds, ratios, expected responses, and response-shape templates based on the measured profiles.

In some embodiments, the signal-processing circuit produces a feature vector rather than a single scalar magnitude. The feature vector may include operating-frequency magnitude, diagnostic-frequency magnitude, in-phase value, quadrature value, phase, ratio, slope, area, peak width, peak count, ring-down amplitude, ring-down decay time, diagnostic-window value, offset-compensated value, comparator state, saturation state, gain state, temperature, pressure, tubing identifier, set identifier, door state, latch state, valve state, pump state, or combinations thereof. A rules-based classifier, lookup table, statistical classifier, or machine-learning classifier may classify the feature vector as fluid, air, foam, particulate, absent tube, empty tube, misloaded tube, bridge, contamination, sensor fault, or low-confidence state.

In some embodiments, the bridge-diagnostic operation may be performed with a single transmitter and receiver, a transceiver pair that alternates transmit and receive roles, multiple transmitters, multiple receivers, an upstream acoustic path, a downstream acoustic path, aligned acoustic paths, anti-aligned acoustic paths, angled acoustic paths, or redundant acoustic paths. Agreement or disagreement among acoustic paths may be used to localize the bridge, distinguish bridge material from in-lumen fluid, estimate bubble position, or validate a bridge-clearing event.

In some embodiments, a bridge-diagnostic result is logged with time, date, sensor identifier, set identifier, operating frequency, diagnostic frequency, bridge metric, expected conduit state, pump state, pressure, temperature, user action, clearing result, alarm status, and therapy status. The log may support service diagnostics, quality tracking, lot investigation, preventive maintenance, field verification of bridge-related alarm generation, or evaluation of conditions that could make air-in-line detection unreliable.

In some embodiments, the controller need not identify a chemical composition of the bridge-forming material. The controller may instead classify the external acoustic bridge based on response inconsistency between an expected conduit state and one or more ultrasonic features, including off-band magnitude, phase, ring-down, decay time, quality factor, diagnostic-window offset, frequency-response shape, disagreement among multiple acoustic paths, or combinations thereof.

In some embodiments, the diagnostic frequency corresponds to a local peak, local minimum, shoulder, slope region, phase-transition region, ring-down region, or other portion of a frequency response that is more sensitive to an external bridge than to liquid within the intended internal lumen. In some embodiments, different diagnostic frequencies are selected for different tubing sizes, tube materials, sensor lots, disposable-set identifiers, or expected therapies.

The described embodiments are illustrative and non-limiting. Features described in connection with one embodiment may be combined with features described in connection with another embodiment unless such combination is technically incompatible. The words “about,” “approximately,” and similar terms encompass ordinary engineering tolerances and measurement variation. The terms “first,” “second,” and similar terms are used to distinguish elements and do not require a particular order unless expressly stated. The term “comprising” does not exclude additional elements or steps.

What is claimed is:

1. A fluid-line monitoring system, comprising:
a fluid conduit having an internal lumen through which fluid flows;
an ultrasonic transducer arrangement positioned relative to the fluid conduit so that an intended acoustic path for air-in-line detection extends through the internal lumen;
a signal-processing circuit configured to generate response data from a received ultrasonic signal; and
a controller configured to:
operate the ultrasonic transducer arrangement under a sensing condition to generate first response data for detecting air in the internal lumen;
operate the ultrasonic transducer arrangement under a bridge-diagnostic condition to generate diagnostic response data;
determine, from the diagnostic response data, that an external acoustic bridge is present, the external acoustic bridge providing an acoustic bypass path along or around an exterior surface of the fluid conduit instead of through the internal lumen;
generate an alarm condition indicating that air-in-line detection is unreliable in response to determining that the external acoustic bridge is present; and
prevent the first response data from being used as a valid air-clear indication while the alarm condition remains active.

2. The fluid-line monitoring system of claim 1, wherein the ultrasonic transducer arrangement comprises a transmitter and a receiver positioned on opposite sides of the fluid conduit.

3. The fluid-line monitoring system of claim 1, wherein the sensing condition comprises an operating frequency selected for discriminating liquid in the internal lumen from gas in the internal lumen.

4. The fluid-line monitoring system of claim 3, wherein the bridge-diagnostic condition comprises a diagnostic frequency different from the operating frequency.

5. The fluid-line monitoring system of claim 4, wherein the diagnostic frequency is selected to distinguish acoustic coupling through the internal lumen from acoustic coupling along or around the exterior surface of the fluid conduit.

6. The fluid-line monitoring system of claim 1, wherein determining that the external acoustic bridge is present comprises evaluating a bridge metric based on at least one of a diagnostic-frequency magnitude, a ratio between diagnostic and sensing response magnitudes, a phase value, a decay-time value, a frequency-response shape, or agreement between multiple acoustic paths.

7. The fluid-line monitoring system of claim 1, wherein the controller is configured to perform the bridge-diagnostic condition during an expected empty-tube condition.

8. The fluid-line monitoring system of claim 7, wherein the expected empty-tube condition is indicated by one or more of a pump-door state, a tube-loading state, a latch state, a flow-stop state, an absent-tube state, an unprimed-tube state, a priming state, a purge state, or a service-test state.

9. The fluid-line monitoring system of claim 1, further comprising memory storing calibration data for at least one of the sensing condition, the bridge-diagnostic condition, or a bridge criterion used to determine that the external acoustic bridge is present.

10. The fluid-line monitoring system of claim 1, wherein the signal-processing circuit is configured to generate in-phase signal data and quadrature signal data from the received ultrasonic signal, and wherein the controller is configured to compute a magnitude M according to M=√((I_{N}−I_{P})²+(Q_{N}−Q_{P})²), where I_{N} is an instantaneous in-phase component, I_{P} is a baseline in-phase component, Q_{N} is an instantaneous quadrature component, and Q_{P} is a baseline quadrature component.

11. The fluid-line monitoring system of claim 10, wherein the signal-processing circuit comprises an active-rectification circuit, first and second integrators, and sample-and-hold circuits configured to sample primary response windows and diagnostic response windows.

12. The fluid-line monitoring system of claim 1, further comprising a sensor housing having a tube-receiving groove configured to register the fluid conduit relative to the ultrasonic transducer arrangement.

13. The fluid-line monitoring system of claim 12, wherein the sensor housing includes a drainage feature, vent, weep channel, hydrophobic surface, oleophobic surface, low-surface-energy coating, acoustic isolation gap, or relief channel positioned to reduce retained bridge-forming material along the exterior surface of the fluid conduit.

14. The fluid-line monitoring system of claim 1, wherein the alarm condition comprises a bridge alarm, a sensor-fault alarm, or an air-detection-unreliable alarm.

15. The fluid-line monitoring system of claim 1, wherein the controller is configured to maintain the alarm condition until a bridge-clearing test indicates that the external acoustic bridge is no longer present.

16. A method of operating a fluid-line monitoring system, comprising:
transmitting ultrasonic energy toward an internal lumen of a fluid conduit through which fluid flows;
generating first response data for detecting air in the internal lumen;
generating diagnostic response data under a bridge-diagnostic condition;
determining, from the diagnostic response data, that an external acoustic bridge is present, the external acoustic bridge providing an acoustic bypass path along or around an exterior surface of the fluid conduit instead of through the internal lumen;
generating an alarm condition indicating that air-in-line detection is unreliable in response to determining that the external acoustic bridge is present; and
withholding a valid air-clear indication while the alarm condition remains active.

17. The method of claim 16, wherein generating the diagnostic response data comprises driving an ultrasonic transducer arrangement at a diagnostic frequency different from an operating frequency used to generate the first response data.

18. The method of claim 16, further comprising maintaining the alarm condition until a bridge-clearing test indicates that the external acoustic bridge is no longer present.

19. The method of claim 16, further comprising inhibiting pumping, preventing therapy from starting, stopping infusion, prompting cleaning, prompting tube reloading, or storing a diagnostic log entry in response to the alarm condition.

20. A medical pump for delivering fluid through a tube, comprising:
a pump mechanism configured to move fluid through the tube;
an ultrasonic air detector positioned relative to the tube so that an intended acoustic path for detecting air extends through an internal lumen of the tube; and
a pump controller configured to:
perform a bridge-diagnostic operation for detecting acoustic coupling along or around an exterior surface of the tube;
determine that an external acoustic bridge is present based on the bridge-diagnostic operation;
generate an alarm condition indicating that air-in-line detection is unreliable when the external acoustic bridge is present; and
prevent a fluid-present response from being used as an air-clear indication while the alarm condition remains active.

21. The medical pump of claim 20, wherein the pump controller is configured to select one or more bridge-diagnostic parameters based on one or more of a flow-stop identification signal, an optical identifier of a disposable set, a keyed geometry of the disposable set, a pump-door state, a latch state, or a carriage state.
